(19) United States
(12) Patent Application Publication
(10) Pub. No.: US 2004/0009507 A1
(43) Pub. Date: Jan. 15, 2004

(54) Concatenated nucleic acid sequence

(75) Inventors: Gregory Paul Winter, Cambridge (GB); Laurent Jespers, Cambridge (GB); Ignace Lasters, Antwerpen (BE); Peter Wang, Vallejo, CA (US)

Correspondence Address: PALMER & DODGE, LLP, KATHLEEN M. WILLIAMS, 111 HUNTINGTON AVENUE, BOSTON, MA, 02199, US

(73) Assignee: Domantis, Ltd.

(21) Appl. No.: 10/412,382

(22) Filed: Apr. 11, 2003

Related U.S. Application Data
(63) Continuation of application No. PCT/GB01/04615, filed on Oct. 15, 2001.

(30) Foreign Application Priority Data
Oct. 13, 2000 (GB) 0025144.7

(51) Int. Cl. C12Q 1/68; C07H 21/04; C12P 19/34

(52) U.S. Cl. 435/6; 435/91.2; 536/24.3

(57) ABSTRACT

An in vitro method for constructing a concatenated head-to-tail repertoire of target nucleic acid sequences is revealed. In particular, the method relates to cycles of concatenation whereby after a single cycle of concatenation, not more than two identical copies of each target nucleic acid sequence are linked together head-to-tail on the same molecule of DNA. The present method ensures that each molecule of a concatenated repertoire is derived from a single template target sequence of the starting repertoire.

### BACKGROUND OF THE INVENTION

1. Field of the Invention

The present invention relates to a method for the production of concatenated head-to-tail molecules from target nucleic acid sequences. In particular, the invention relates to an in vitro concatenation method for generating concatenated molecules from a repertoire of target nucleic acid sequences such that after each concatenation cycle, not more than two identical copies of each target nucleic acid sequences are linked together head-to-tail on the same molecule of DNA.

2. Description of the Related Art

Combinatorial repertoires, produced through either genetic or synthetic means, have been developed as a tool to rapidly select or to screen for molecules of interest (such as (ant)agonists, inhibitors, antibodies, enzymes, and other polypeptides). These repertoires are particularly useful to circumvent the limitations of rational design approaches, such as the lack (or the absence) of structural information about the target molecule and the limited capacity of computer software to model molecular complexes, three-dimensional structures of proteins and active sites of enzymes.

Combinatorial repertoires consist of degenerate populations of polymers which use nucleotides, amino acids, carbohydrates or synthetic molecules as building blocks. Since each polymer of a combinatorial repertoire is generated by sequential addition of randomly chosen building blocks, the theoretical molecular diversity of these repertoires can be calculated from the number of combinatorial positions within a polymer and from the number of building blocks that can be proposed at each combinatorial positions. Thus a totally randomised protein 100 amino acids in length can contain 20¹⁰⁰ different amino acid sequences.

Practically, such huge molecular diversity cannot be sampled in the laboratory: for example, the largest phage display repertoires contain 10¹⁰-10¹¹ individual clones, and in vitro based combinatorial repertoires based on transcription/translation of DNA do not exceed 10¹³ molecules. The limited size of these repertoires is however not always critical, provided that the starting combinatorial repertoire is reasonably close to some intended target, or to use other words, provided that the distance between the starting and the intended target sequences is not larger than the sub-sequence space defined by all the molecules created in the repertoire. For example, the introduction of additional sequence variability, through error-prone PCR (a method which mimics the molecular evolution of proteins by point mutations) has been used to increase the affinity of antibodies for their antigens (e.g. Hawkins et al., 1992; Gram et al., 1992; Daugherty et al., 2000), or the catalytic activity/stability of enzymes (e.g. You et al., 1996; Cherry et al., 1999; Song & Rhee, 2000). In contrast, evolving a poly-alanine sequences into a functional enzyme such as triose phosphate isomerase would require the exploration of a sequence space of an unmanageable size.

Selection from combinatorial repertoires is to some extent reminiscent of the selection of the fittest species in the process of natural molecular evolution. Since this process has clearly been able cope with an almost infinitely larger sequence variation than is found in present combinatorial repertoires, strategies other than point mutation must have contributed to the rapid generation of protein diversity.

One process, homologous recombination, involves shuffling homologous genes via cross-overs between chromosome pairs. Following this observation, computer simulations have shown the importance of iterative combinatorial rearrangements for protein evolution (e.g. Arkin & Youvan, 1992; Bogarad & Deem, 1999). Indeed homologous recombination alleviates the theoretical and practical problems faced by point mutation-based approaches, by reassembling related genes which have been “pre-filtered” for the appropriate properties. Recently, DNA techniques such as DNA shuffling (Stemmer, 1994) and STEP (Zhao et al., 1998) have proven successful in simulating such recombination events at the level of a single genes: in these processes, multiple related genes (either generated by error-prone PCR or naturally occurring) are used as parental sequences (encoding enzymes, antibodies and operons). Randomly-generated fragments of these genes are reassembled to generate a progeny of chimaeric genes which are then selected or screened for a desired property and/or submitted to another round of DNA recombination. While this method works efficiently to rapidly improve gene function, the use of a pool of related genes as breeding material makes it inadequate to evolve protein diversity from distantly-related building blocks.

Both from a structural as well from a genetic point of view (exons), it has become apparent that proteins are assembled from a repertoire of unit blocks: secondary structures (helices and strands), supersecondary structural motifs, globular domains and oligomers. Often, the three-dimensional organisation of these elements follows rules of symmetry (for review, see Blundell & Srinivasan, 1996; Wolynes, 1996). In viruses, the restriction imposed on size of the genetic material may have been the driving force for generating symmetrical viral coats made of repeated subunits encoded by a few genes.

Homodimerisation is a commonly observed phenomenon amongst proteins involved in cell signalling (hormones, receptors, etc.) and in immunity (such as immunoglobulins and receptors). It should be noted that, since amino acids are synthesised as L-enantiomers only, identical structural elements can only be related to each other via rotational symmetries (e.g. two-fold for homodimeric receptors). In single non-oligomeric proteins, symmetries have also been observed in the tertiary structure although the amino acid sequences corresponding to the symmetrical elements of the structure may not be identical. For example, aspartic proteinases (e.g. pepsin) in higher organisms are composed of two globular domains oriented toward each other according to a quasi two-fold rotational symmetry. The strong structural homology of these enzymes with the homodimeric HIV proteinase suggests that the ancestor of the pepsin family arose through gene duplication of a double-psi-barrel fold similar to that of the HIV proteinase followed by fusion and genetic drift over time (Lapatto et al., 1989). Recently, Coles et al. (1999) proposed an evolutionary path whereby the double-psi-barrel fold would have itself arisen by duplications and permutation of an ancestral 40-residue βαββ element. In another example, the evolution of the α/β barrel proteins would have been driven by the recurrent use of a single α/β supersecondary unit (Lonber & Gilbert, 1985; Fraber & Petsko, 1990; Rackovsky, 1998; Lang et al., 2000). Computer simulations of protein folding have also emphasised the role of symmetries in single non-oligomeric polypeptide sequences, as a useful means to generate a funnelled energy landscape (Wolynes, 1996). Indeed, for reasons of symmetry, tandem repeats of building blocks are more likely to yield a folded protein if (a) the monomeric unit block has a high propensity to adopt some stable (super)secondary structure, and/or (b) the monomeric unit block forms some favourable inter-unit interactions.

Taken together, these observations emphasise how partial or entire gene duplication may have been a critical factor next to point mutation and homologous recombination in the generation of protein diversity. They have also prompted de novo design experiments where novel α/β barrels or four-helix bundles are assembled by genetic fusion of tandemly repeated building blocks (e.g. Goraj et al., 1990; Houbrechts et al., 1995; Hecht et al., 1990; Regan & DeGrado, 1988; Schafmeister et al., 1997).

The process of gene multimerisation circumvents the combinatorial explosion problem described above. Indeed, in concatenated polypeptide repertoires, the encompassed sequence space does not exceed that of the basic polypeptide unit, even though each member may contain large number of positions targeted for mutation. The potential usefulness of such concatenated polypeptide repertoires can be illustrated by calculating the probability that a given 5 amino acid element will occur independently in two positions in a single randomised 30-residue sequence (about 1 in −3×10¹⁰). If the randomised 30-residue sequence is replaced with a repertoire of two concatenated 15-mers, then this probability improves by about 5 orders of magnitude (about 1 in 3×10⁵). Depending on the type of basic polypeptide unit and the number of repeats in the concatenated polypeptide, these combinatorial repertoires can be viewed either as protein repertoires of variable intended architecture, or peptide repertoires of possible high-binding avidity.

The earliest attempts to clone DNA fragments in the form of concatenated copies involved self-ligation of DNA fragments (either blunt-ended or carrying complementary cohesive ends) yielded poor results due to the random orientation of fragments in the resulting clones (Hardies et al., 1979; Sadler et al., 1980). Interestingly, it was observed that the presence of inverted repeats in a multimer lead to instability, whereas a series of direct repeats would form stable clones in bacteria (Sadler et al., 1978). Control of fragment orientation was subsequently achieved by Hartley and Gregori (1981) using DNA fragments carrying distinguishable cohesive ends. As a result, only head-to-tail combinations result in perfect matches and therefore substrates for DNA ligation. The asymmetric and complementary ends can be generated by restriction endonucleases (such as AvaI; Hartley & Gregori, 1981), and class IIs-restriction enzymes (Kim & Szybalski, 1988) but also via ligation of tailored adapters (Taylor & Hagerman, 1987).

Polymerase chain reaction (PCR)-based approaches have been proposed for the generation of concatenated DNA sequences (White et al., 1991; Jiang et al., 1996; Shiba et al., 1997). These methods result in the concatenation of the oligonucleotide primers but without control of the number of fragments per concatamer. Moreover, spurious insertion or deletion of a few nucleotides has been reported at the junctions (Shiba et al., 1997). More importantly, these methods are not suitable for the concatenation of repertoires since thermal denaturation of the double-stranded DNA fragments will invariably result in scrambling of the DNA units within the concatenated products.

To control both the orientation and the number of fragments per concatamer, Cohen and Carmichael (1986) proposed a method wherein each target nucleic acid sequence would have a unique pair of cohesive ends, thereby allowing simultaneous ligation of all units into a vector. Other groups have followed a sequential approach wherein a fragment of n-units was obtained by several cycles of restriction/ligation/transformation using the resulting concatenated units as DNA fragments for ligation into the vectors recovered after transformation (Hofer, 1987; Goraj et al., 1990). Unless they are performed separately for each targeted fragment (which is technically impossible for large repertoires), these methods are however not amenable for concatenation of large collection of target nucleic acid sequences, in which ligation would concatenate target nucleic acid sequences of the same sequence only.

To date, only one approach has tackled the concatenation issue for repertoires by using a rolling replication approach on short DNA circles (Fire & Xu, 1995). It involves four steps: (1) self-ligation of a template oligonucleotide encoding the randomised target nucleic acid fragment using a partially complementary oligonucleotide as guide; (2) extension of the complementary strand of the circularised template oligonucleotide from the annealed complementary oligonucleotide using a nucleic acid polymerase in the presence of triphosphate precursors; (3) displacement of the neo-synthesised strand by the polymerase, leading to rolling circle replication; (4) synthesis of the complementary strand of the extended strand followed by cloning. In 10 of 18 clones, all targeted fragments are oriented in the same orientation (head-to-tail) and represent 3-5 tandem copies derived from individual template sequences. However, it should be pointed out that a cycle of concatenation generates a wide range of extensions from the annealed primer which prevents control of the number of concatenated copies of target nucleic acid sequences in each clone of the resulting repertoire (Fire & Xu, 1995; Brown, 1997). Such variability is not desirable in a repertoire of concatenated polypeptides for at least two reasons: (1) the diversity of the “useful” repertoire for screening or selection is greatly reduced since it comprises a vast majority of clones that do not comprise the optimal number of concatenated target nucleic acid sequences; for example, a concatenated repertoire aiming at creating novel TIM-like barrel proteins should essentially comprise polypeptides sequences carrying 8 copies of a single putative α/β unit since this is the only geometrically acceptable number of repeats which is compatible with such a protein fold. In another example, duplicated polypeptide sequences are putatively the only and most appropriate source of specific polypeptides recognising homo-dimeric molecules such as cell-receptors; (2) a repertoire of concatenated polypeptides that is not homogeneous as to the number of concatenated unit per clone is more likely to generate artefacts if, for example, selection or screening is oriented for binding activity to a bait molecule immobilised on a solid support. Indeed, avidity effects may favour the isolation of high-copy multimers in contrast to multimers carrying the intended number of copies.

Overall, all methods described above but one (the method using class-II restriction enzymes) also suffer from the fact that the boundaries of each of the target nucleic acid fragments of a concatamer are pre-determined either by the requirement of a restriction site, the ligation of an adaptor, or the hybridisation of an extension primer. In the latter example, about ten base pairs at each ends of the target nucleic acid fragments must be kept constant to allow annealing of the extension primer, which results in the presence of an invariant hexapeptide motif between each unit upon translation. This considerably complicates the design of repertoires encoding symmetrical proteins, and also reduces the sequence space diversity of permutated clones.

### SUMMARY OF THE INVENTION

A major failing in the prior art, which is not solved or indeed sought to be solved by any of the methods identified above, is that the concatenated repertoires produced have not been homogenous—that is, they have contained varying numbers of duplications of the target sequence. We have determined, as described herein, that homogeneity is a desirable attribute in a concatenated repertoire, which provides a number of advantages. The method of the present invention, for the first time, allows the generation of homogenous concatenated repertoires.

The present invention is directed to a novel method for the production of head-to-tail concatenated nucleic acid sequences and their encoded polypeptides. This method is suitable for the generation of repertoires of head-to-tail concatenated nucleic acid sequences from which the desired nucleic acid fragment may be isolated by selection or screening of the nucleic acid-encoded product. The present method does not have any of the limitations described in the above methods. In particular, the method allows the concatenation of a repertoire of nucleic acid sequences such that after one or several concatenation cycles, the resulting repertoire is substantially homogeneous.

According to a first aspect, therefore, there is provided a homogenous repertoire of concatenated nucleic acid sequences.

Such a repertoire may be considered to be a repertoire of concatenated nucleic acid sequences wherein not more than two identical copies of each target nucleic acid sequence are linked together in head-to-tail orientation on the same molecule of DNA. Where such a repertoire is the subject of multiple concatenation cycles, it can be understood that the “target nucleic acid sequence” should be considered to be the sequence resulting from the penultimate concatenation cycle.

Although concatenated DNA molecules are known in the art, and attempts to create repertoires thereof have been made, the invention provides, for the first time, the ability to create homogenous repertoires in which substantially each concatenated nucleic acid molecule has the same number of target nucleic acid sequences. In order to achieve this, each round of concatenation produces concatamers of exactly two sequences. This allows the total number of sequences in each concatamer to be precisely controlled.

The invention moreover provides a repertoire of concatenated polypeptides encoded by the concatenated nucleic acid sequences.

In a second aspect, the invention provides a method for creating a concatenated repertoire of target nucleic acid, wherein not more than two identical copies of each target nucleic acid sequence are linked together in head-to-tail orientation on the same molecule of DNA.

The invention provides a method for concatenating a target nucleic acid sequence such that after a single cycle of concatenation not more than two copies of the target nucleic acid sequence are linked together head-to-tail on the same molecule of double stranded DNA (e.g. a double stranded replicon). Preferably, each of two complementary strands of DNA of a target nucleic acid sequence is used as template for synthesis of a complementary strand of nucleic acid so as to generate not more than two copies of each of the target nucleic acid sequences, which are subsequently ligated together in a head-to-tail orientation on the same molecule of DNA.

Preferably, the target nucleic acid sequences are incorporated into double-stranded replicons, such as plasmids, cosmids or bacteriophage vectors.

Advantageously, the method according to the invention involves introducing two single-strand nicks, one at each of the 5′ ends of the target nucleic acid sequence, such that the top and bottom strands of the target nucleic acid sequence are converted into 5′-overhangs; incubating the resulting nicked DNA sequence with a nucleic acid polymerase under conditions which result in filling of the 5′-overhangs to, generate blunt ends (thereby creating two identical copies of the target nucleic acid sequence on the same molecule of DNA); and incubating the resulting blunt-ended DNA sequence with a nucleic acid ligase to covalently link the two copies of the target nucleic acid sequence in a head-to-tail orientation).

In an advantageous aspect, several cycles of concatenation can be performed, wherein the target nucleic acid sequence of a further cycle includes the product of a previous cycle of concatenation; such that after each concatenation cycle, the DNA product (and its encoded polypeptide) comprises a head-to-tail duplication of the nucleic acid sequence (and its encoded polypeptide sequence) targeted in each concatenation cycle, respectively.

Any suitable number of concatenation cycles, from 1 up to 5, 6, 7, 8, 9, 10 or more may be performed. Preferably, 1, 2, 3 or 4 concatenation cycles are performed.

In a further advantageous aspect, the invention as described above provides a means by which concatenated copies of a collection of many different target nucleic acid sequences (i.e. a repertoire) can be generated such that each concatenated nucleic acid sequence results from the concatenation of a single target nucleic acid molecule from the starting repertoire.

Concatenated nucleic acid molecules generated by a method according to the invention may be further manipulated, such as by amplification of selection, in order to achieve a desired end. Thus, for example, concatenated nucleic acid molecules in a ligated repertoire according to the invention may be amplified by transformation of a host cell with said repertoire. Moreover, the invention envisages amplifying the ligated repertoire of target nucleic acid sequences by polymerase chain reaction with oligonucleotide primers encompassing the target nucleic acid sequences, purification of the amplified DNA product, cloning into a double-stranded replicon, and transformation of a host cell with the ligated product.

The nicks introduced into the target nucleic acids are advantageously introduced in replicon sequence, thus defining the target nucleic acid sequence as that sequence, in a nucleic acid, which is between the nicks formed in the top and bottom strands thereof.

Preferably, the nicks are introduced by a site-specific nicking endonuclease. Single-stranded nicking endonucleases, sometimes referred to as nickases, are well known in the art. Numerous examples of such enzymes may be identified, for example, by searching on appropriate databases such as GenBank. preferred examples of such endonucleases include N.BstNBI. Further examples are set forth below.

The polymerase enzyme employed in the present invention advantageously displays strand-displacement activity. Examples of polymerases which display such an activity include the Klenow fragment of DNA polymerase I, phage Phi29 DNA polymerase, Vent DNA polymerase, and Vent (exo⁻) DNA polymerase. Single-strand DNA-binding proteins such as E. coli SSB and T4 gene 32 protein may advantageously be used in combination with the polymerase to facilitate the filling of the 5′-overhangs. Alternatively, conversion of the nicked DNA into a blunt-ended DNA molecule can be performed with thermophilic DNA polymerases such as Vent DNA polymerase, Vent (exo⁻) DNA polymerase, or Bst DNA polymerase (large fragment) provided that the DNA has not been completely denatured prior to the elongation step, and provided that the elongation is taking place is at a temperature which is suitable for unpairing of the cohesive-ends but reasonably lower than the melting temperature of the whole replicon which contains the targeted nucleic acid sequence.

In a still further aspect, the invention provides a method for preparing concatenated polypeptides according to the invention, comprising the steps of:

a. creating a concatenated repertoire of target nucleic acid sequences by a method according to the invention;

b. translating the concatenated repertoire of target nucleic acid sequence to produce a repertoire of encoded concatenated polypeptides;

c. screening the encoded concatenated polypeptides for possession of a desired activity.

In an advantageous embodiment designed for screening of the repertoire and selection of peptides having desired activities, each encoded concatenated polypeptide of the repertoire is expressed as a fusion protein. Preferably, it is expressed fused to a surface component of an organism so that each organism in a population thereof displays a concatenated polypeptide at its surface and encapsidates a concatenated nucleic acid encoding the displayed concatenated polypeptide within. Preferably, the organism is a bacteriophage.

Concatenated nucleic acids selected according to the invention are advantageously used to express a concatenated polypeptide in a host cell. Alternatively, the translated sequence of concatenated nucleic acid may be used to derive a polypeptide by chemical synthesis.

The nucleic acids and/or polypeptides of the invention may moreover be further manipulated at the nucleic acid or protein level. For example, they may be manipulated by a technique selected from the group consisting of mutagenesis, fusion, insertion, truncation and derivatisation. Such techniques are known to those skilled in the art.

### BRIEF DESCRIPTION OF THE FIGURES

FIG. 1 depicts the approach described in the present invention for head-to-tail duplication of a repertoire of target nucleic acid sequences. 1.a. The repertoire forms a targeted region within a collection of double-stranded DNA molecules of which both ends are physically linked together (e.g. in a replicon such as a plasmid or phage). 1.b. Incubation of the DNA repertoire with a site-specific nicking endonuclease aiming at introducing two single-strand nicks, one at each of the 5′ ends of the target nucleic acid sequence, such that the top and bottom strands of the target nucleic acid sequence are converted into 5′-overhangs;) 1.c. Filling of the 5′-overhangs by incubation with a nucleic acid polymerase in the presence of nucleotide triphosphates, thereby generating two double-stranded DNA copies of each target nucleic acid sequences, which are held in closer proximity. than that of any two non-identical copies of target nucleic acid sequences. 1.d. Head-to-tail attachment of the resulting blunt ends with DNA ligase Here, the physical link between the top and bottom strands of each molecule of the repertoire ensures that, upon ligation of the filled overhangs, each concatenated molecule is most likely to be derived from a single target nucleic acid sequence of the repertoire. 1.e. Steps 1.b to 1.d. represent a single concatenation cycle, which can be repeated with the product (either in its entire length or on a dedicated fragment) of a previous concatenation cycle to generate concatenated molecules of increasing lengths.

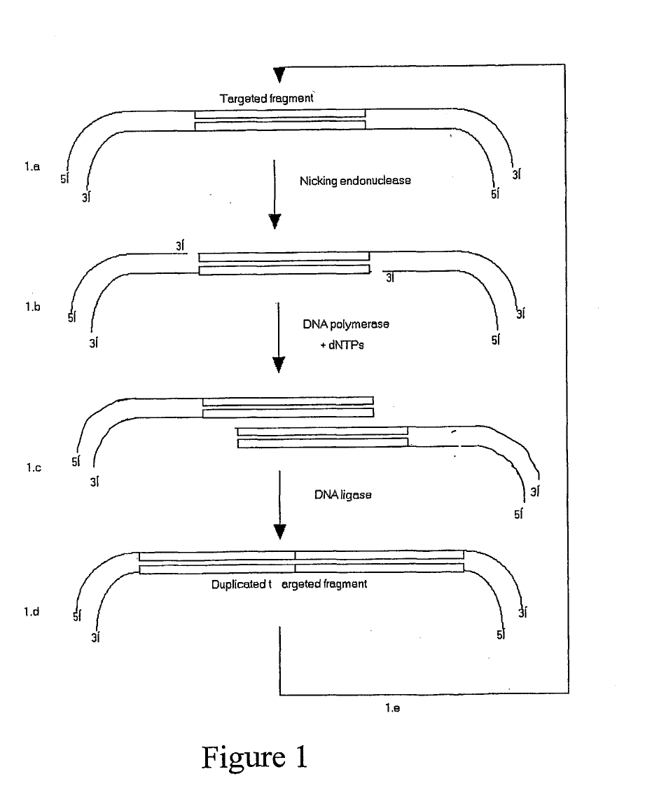

FIG. 2.a shows the DNA sequence of the 84-bp DNA fragment encompassing the randomised V_{H}-CDR2, that was cloned into pK4. Relevant restriction and nicking sites are shown. FIG. 2.b shows the DNA sequences (and relevant restriction sites) of seven positives clones obtained after one duplication cycle. The underlined nucleotides are those targeted for randomisation in the repertoire. The symbol “*” represents a nucleotide deletion.

FIG. 3. Shows the digestion of pK4-V_{H}-CDR2 plasmid DNA before and after each of the four cycles of concatenation, with restriction enzymes BamH1/KpnI (lanes 2 to 6) or SpeI (lanes 9 to 13). Lanes: 2 & 9 loaded with pK4-V_{H}-CDR2; 3 & 10 loaded with pK4-(V_{H}-CDR2)₂; 4 & 11 loaded with pK4-(V_{H}-CDR2)₄; 5 & 12 loaded with pK4-(V_{H}-CDR2)₈; 6 & 13 loaded with pK4-(V_{H}-CDR2)₁₆. Expected lengths of shorter molecular weight products after BamH1/KpnI digestion: lane 2: 192 bp; lane 3: 276 bp; lane 4: 444 bp; lane 5: 780 bp; lane 6: 1,452 bp. Number of SpeI sites in lane 9: none; lane 10: 1, lane 11: 3 (resulting in two 84-bp fragments per plasmid molecule); lane 12: 7 (resulting in six 84-bp fragments per plasmid molecule); lane 13: 15 (resulting in fourteen 84-bp fragments per plasmid molecule). Lane 1: phage Lambda/HindIII restriction digest. Lane 8: phage PhiX174/HaeIII restriction digest.

FIG. 4.a shows the 45-bp target nucleic acid sequence encoding the 15-mer peptide repertoire, that was cloned into Fd-Tet-SN. Relevant restriction and nicking sites are shown. FIG. 4.b shows the peptide sequences of eight positives clones obtained after one cycle of concatenation. Seven products carry two copies of the target nucleic acid sequences, whereas the eighth clone (n° 3) contains a mixed 90-bp sequence which most likely results from spurious ligation of two linearised vectors. FIG. 4.c. shows the peptide sequences of twelve clones obtained after cycles of concatenation. Eight products carry four copies of the 45-bp target nucleic acid sequence, whereas the four others (n° 3, 4, 9 and 11) contain a mixed 180-bp sequence which most likely results from spurious ligation of two linearised vectors. The symbol “*” indicates that an amber codon was found at that position: in E. coli TG1 cells, this would encode for a glutamine residue.

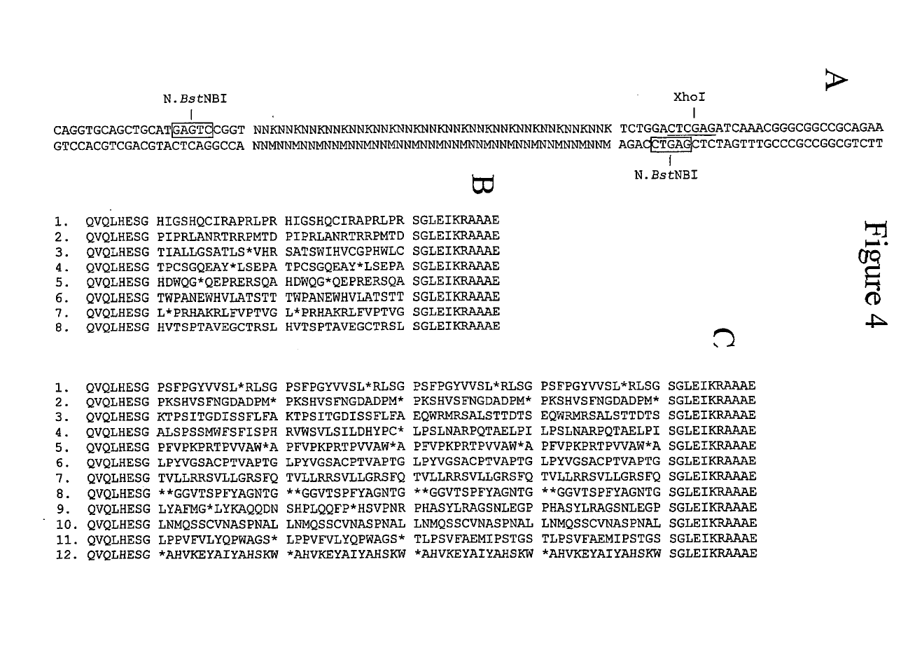

FIG. 5 shows the binding properties of single alanine-mutants of MP to EC-EpoR-Fc. Each mutant was produced as a phage-displayed peptide and analysed for binding to EC-EpoR-Fc by phage-ELISA. From the dilution series and a control curve obtained with wild-type MP displayed on phage, the binding affinity of each mutant phage was expressed in percent of the binding activity of wild-type MP.

FIG. 6 represents the MP peptide according to a quasi twofold rotational axis of symmetry, in order to pinpoint the functional equivalence (or non-equivalence) between pairs of residues from each tandem repeat. Squares and circles represent residues which have been or not been targeted for alanine-scanning mutagenesis, respectively. The shading in squares are proportional to the extent of which a given residue is critical or not for EC-EpoR-Fc binding.

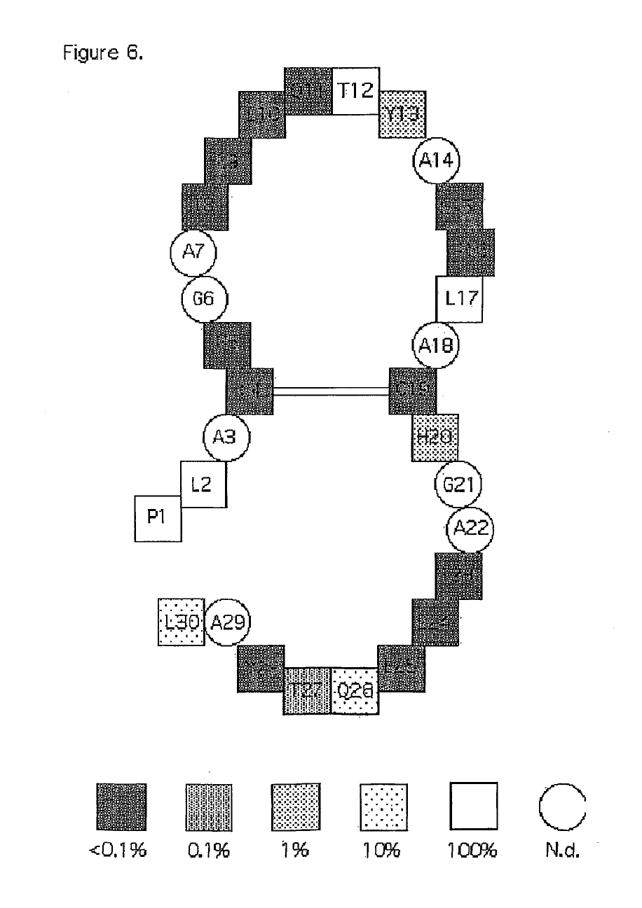

FIG. 7 shows the products of concatenation of biotinylated linear DNA fragments attached to streptavidin beads. Lanes 1, 3, and 5 show PCRs of untreated beads carrying clone 6.1, 6.2, or both. Lanes 2, 4, and 6 show PCRs of beads carrying clone 6.1, 6.2, or both that were treated with one cycle of concatenation (nicking, extension, ligation). Lane 7 is a molecular weight standard (φX174 HaeIII digest).

### DETAILED DESCRIPTION OF THE INVENTION

Definitions

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

In the context of the present invention, a “target nucleic acid sequence” refers to any nucleic acid sequence which forms the substrate for a cycle of concatenation. A target nucleic acid sequence is a double-stranded DNA molecule which may be of natural or synthetic origin. Advantageously, the target nucleic acid sequence is incorporated into a double-stranded replicon such as a plasmid, a phagemid, a phage or a cosmid. The target nucleic acid sequence is in practice defined by the location of the single-stranded nicks made in the replicon or other DNA molecule in which the sequence is found. Thus, the target nucleic acid sequence is advantageously a subsequence of a larger target nucleic acid molecule. The target nucleic acid sequence is preferably double stranded and may be any number of nucleotides in length. For example, but not essentially, it may be a discrete number of nucleotide triplets in length, such as 6, 9, 12, 15, 18, 21, 24, 27, 30, 33, 36, 39, 42, 45, 90, 120 or 150 nucleotides in length, or more. Advantageously, it is between 9 and 672 nucleotides in length.

A “target nucleic acid molecule” is a nucleic acid molecule which comprises the target nucleic acid sequence. Advantageously, this molecule may be a replicon, such as a phage, plasmid, phagemid, chromosome or other vehicle. It is advantageously circular. However, it may also be a linear nucleic acid molecule, for example immobilised on a solid substrate. It is an advantageous feature of the target nucleic acid molecule that the ends thereof distal to the target nucleic acid sequence are unligatable, for instance as a result of the circularity of the molecule or as a result of immobilisation onto a solid surface.

The term “starting repertoire” refers to a collection of many unique target nucleic acid sequences, which may be created by any suitable means in any form, and are the substrate for a cycle of concatenation. Sequence differences between repertoire members are responsible for the molecular diversity present in the starting repertoire. Advantageously, the starting repertoire is incorporated into a collection of double-stranded replicons such as a plasmid, a phagemid, a phage or a cosmid. Preferably, the double-stranded replicons allow expression of the target nucleic acid sequences into the corresponding polypeptides. Alternatively, the repertoire may be immobilised or arrayed on to a solid support.

Repertoires according to the invention advantageously comprise a plurality of members, typically comprising between 10 and 10¹³ different target nucleic acid molecules. Advantageously, repertoires comprise between 10⁴ and 10⁸ different target nucleic acid molecules.

In the context of the present invention, a “polypeptide” is a sequence of amino acid residues typically between 3 and 224 amino acid residues, or longer, which is produced by translation of the corresponding nucleic acid sequence. A polypeptide may therefore correspond to a peptide (typically between 3 and 50 amino acids residues) as well as to a folded protein (typically between 50 and 224 amino acids residues, or longer).

The term “cycle of concatenation” refers to the process wherein at least two identical copies of each target nucleic acid sequence are linked together head-to-tail on the same molecule of DNA. The substrate for a cycle of concatenation is the starting repertoire. The rolling replication approach on short DNA circles (Fire & Xu, 1995) provides an example of such cycle of concatenation.

The “concatenated repertoire” is the product of transformation of a starting repertoire by a single cycle of concatenation. Thus, a concatenated repertoire comprises many concatenated target nucleic acid sequences. The concatenated repertoire may, if needed, constitute a new starting repertoire for the next cycle of concatenation to generate a novel concatenated repertoire. This process can be repeated several times if needed. According to the present invention, each cycle of concatenation results in the dimerisation of the target nucleic acid sequences. This means that the repertoires are homogenous, that is they are of controlled concatamer dimensions, and substantially each molecule in the repertoire will comprise the same number of target nucleic acid sequences. As used herein, “homogenous” means that most, though not necessarily all, of the molecules of the repertoire have the same number of target nucleic acid sequences. “Substantially each”, therefore, refers to more than about 50% of the molecules, advantageously more than about 60%, 70%, 80%, 90%, 95%, 98% or 99% of the molecules of the repertoire.

The term “concatenated polypeptide” refers to a sequence of amino acid residues, which may be for example between 6 and 448 amino acid residues, or longer, which is produced by translation of the corresponding concatenated nucleic acid sequence. A concatenated polypeptide may correspond to a disordered peptide (typically between 6 and 50 amino acids residues) as well as to a folded protein (typically between 50 and 448 amino acids residues, or longer).

In the context of the present invention, “encoded” means that each target nucleic acid sequence of a repertoire, whether it is a starting or a concatenated repertoire, is specifically associated, either covalently or via interacting partners (e.g. via display on the surface of an organism such as a bacteriophage), or via compartmentalisation, to its translation product, that is a polypeptide or a concatenated polypeptide. Polypeptide members of an encoded repertoire can be selected or screened for binding or function and further characterised by recovery of the corresponding nucleic acid sequences.

Description

The present invention relates to a method for the production of head-to-tail concatenated nucleic acid sequences and their corresponding concatenated polypeptides. In an advantageous aspect, the invention affords to create a concatenated repertoire of target nucleic acid sequences such that each concatenated target nucleic acid sequence results from the concatenation of a single targeted template molecule from the repertoire. In a further advantageous aspect, the method affords careful control of the number of concatenated copies of each of the target nucleic acid sequences in the concatenated repertoire: in contrast to the rolling-circle replication of short DNA circles (Fire and Xu, 1995), the present invention ensures that the product of a cycle of concatenation, e.g. the concatenated repertoire, is a nucleic acid molecule, or a collection of nucleic acid molecules wherein not more than two copies of each target nucleic acid sequences are linked together in head-to-tail orientation on the same DNA molecule.

The concept at the basis of the present invention is as follows: by dissociating the top and bottom strands of a target nucleic acid sequence, each of the strands can be used as a template for second strand synthesis in the presence of a DNA polymerase, such that after incubation in the appropriate conditions, two copies of said target nucleic acid sequence are generated. Blunt-end ligation of the two copies then generates a head-to-tail duplicated target nucleic acid sequence provided that one copy of said target sequence has an exposed 3′-side and the other copy, an exposed 5′-side. Conversely, one copy of said target sequence must have an unligatable 5′-side and the other copy, an unligatable 3′-side. This can easily be achieved either if both unligatable ends are part of the same circular DNA molecule such as a plasmid, or if both unligatable ends are restricted from free motion, i.e. are immobilised on a solid support. Advantageously, this approach affords to concatenate target nucleic acid sequences of a repertoire, such that each concatenate sequence is derived from a unique template molecule. Indeed, the high-local concentration of two identical copies of each target nucleic acid sequences excludes or at least considerably reduces shuffling upon ligation of copies from different target nucleic acid sequences.

Nicking Enzymes

Taking the target nucleic acid sequence comprised within a circular DNA molecule as example, the substrate for blunt-end ligation is thus a linearised DNA molecule with two copies of the target nucleic acid sequence, one at each end and oriented in the same direction. The strategy outlined in the present invention is reminiscent of recombinant DNA techniques known in the art, aiming at creating new cleavage sites from existing ones after filling in of 5′-overhangs to generate blunt ends which are subsequently ligated to each other (e.g. conversion of EcoRI restriction site into an XmnI restriction site). Thus, provided that 5′-overhangs spanning the entire length of a target nucleic acid sequence can be obtained, second-strand synthesis on both cohesive ends followed by recircularisation of the blunt-ended linearised DNA molecule would predominantly yield duplication of said target sequence Because type IIs restriction enzymes such as BsaI or BbvI recognise asymmetric sites and generate cohesive ends at a defined distance to one side of their recognition sequence, they are a priori appropriate to concatenate a repertoire of target nucleic acid sequences. However, their enzymatic specificities limit the length of the 5′-overhangs to four base pairs at most. 5′-overhangs that extend to several hundreds of base pairs may be created as follows to concatenate a wide range of target nucleic acid sequences. Several strategies are illustrated in this invention, one of these being further illustrated in the following section.

In a preferred embodiment, endonucleases which catalyse single strand breaks at specific sites are preferred. In order to maximise nucleic acid sequence diversity at the 5′-side and 3′-side of the target nucleic acid sequence, nicking endonucleases with similar specificities as type IIs restriction endonucleases are most suitable. One such nicking endonuclease is N.BstNBI which recognises the asymmetric 5′-GAGTC-3′ sequence and catalyses a single strand break four bases beyond the 3′ side of the recognition sequence (New England Biolabs). In all examples described in this invention (see next section), N.BstNBI has been used but it is readily apparent to those skilled in the art that other nicking endonucleases could be used in an identical manner in the invention. In each of the examples outlined in the next section, two recognition sites for N.BstNBI were introduced in opposite direction (3′-side versus 3′-side) at the ends of the target nucleic acid sequence, such that upon incubation with N.BstNBI, 5′-overhangs covering the length of the target nucleic acid fragment were generated. The resulting nicked DNA provides then a substrate for second-strand synthesis to generate two blunt ends. If the two N.BstNBI sites would have been oriented in the 5′-side versus 5′-side, the resulting 3′-overhangs would not be an appropriate substrate for DNA polymerase, unless an oligonucleotide primer would initially be annealed at the 3′-ends of the cohesive ends. This technically workable solution would however impose restrictions on the nucleic acid diversity at the 5′-side and 3′-side of the target nucleic acid sequence. After ligation, the concatenated repertoire can be amplified either by direct transformation of an host or by polymerase chain reaction, and used as a starting repertoire for the next cycle of concatenation, if needed.

In another embodiment, classical type II or type IIs restriction endonucleases can be engineered such that they catalyse single- instead of double-strand breaks. For example, Wende et al. (1996) reported the production and characterisation of an artificial heterodimer of the EcoRV restriction endonuclease. One unit of the EcoRV heterodimer is fully functional while the other is catalytically inactive (by site-directed mutagenesis).

Consequently, the EcoRV heterodimer exhibits single-strand nicking activity at the palindromic recognition site. However, the process is not as optimal as a nicking endonuclease since it does not afford control over which strand is preferentially nicked (thereby yielding a mixture of 5′- and 3′-overhangs) and since it also evolves double-strand DNA cleavage with time due to the association-dissociation equilibrium between the endonuclease and the targeted DNA molecule. Nevertheless, this example shows that type II and preferentially type IIs restriction endonuclease can also be appropriate for the purpose of this invention, provided that adequate site-directed mutagenesis is performed on one unit of these homodimeric enzymes.

In another embodiment, it has long been known that under appropriate conditions and in the presence of the DNA-intercalating agent EtBr, some restriction endonucleases have been shown to nick DNA in one strand rather than cutting both strands of templates containing unique restriction sites (Parker et al., 1977; Osterlund et al., 1982; Kovacs et al., 1984). As described above, the process is again not optimal and will yield a mixture of 5′- and 3′-overhangs, together with a percentage of double-strand DNA breaks. Nevertheless, the use of EtBr with classical restriction enzymes can generate (although at lower efficiency) the appropriate template molecule for concatenation of target nucleic acid sequence.

In another embodiment, Taylor et al. (1985) observed that certain restriction endonucleases (e.g. AvaI, AvaII, NciI) cannot cleave phosphorothioate DNA. As a result, single-strand nicks are generated in DNA containing one phosphorothioate strand and one non-phosphorothioate strand. This observation has formulated the basis for novel methods such as site-directed mutagenesis Nakamaye & Eckstein, 1986) and isothermal DNA amplification by strand displacement (Walker et al., 1992). Thus, provided that phosphorothioate bases are positioned on the appropriate strand of each of the restriction sites encompassing the target nucleic acid sequence, 5′-overhangs can be generated spanning the length of the said target sequence.

Concatenation of Repertoires

The present invention ensures self-concatenation of target nucleic acid sequences even in the context of a repertoire, because in each cycle of concatenation, the two copies of each target nucleic acid sequence are partitioned from the copies of other target nucleic acid sequences before head-to-tail ligation. As described above, one practical way to achieve this is to clone the starting repertoire of target nucleic acid sequences within a collection of replicons such as a plasmid, a phagemid, a phage or a cosmid. Another approach consists in affixing the ends of a collection of linear DNA molecules comprising the target nucleic acid sequences on a solid support.

Preferably, the linear DNA molecules are amplified by polymerase chain reaction using biotinylated oligonucleotide primers, and, after purification, immobilised on a solid phase covered with streptavidin (plastic surface, paramagnetic or polystyrene beads). Scrambling between copies of different target nucleic acid sequences of the starting repertoire would therefore be avoided following to a cycle of concatenation of the repertoire by polymerase chain reaction with the same set of biotinylated oligonucleotide primers, and sizing by electrophoresis on agarose gel (optional), would then generate a novel starting repertoire for a new cycle of concatenation, if needed, using a fresh solid support. In addition to its compatibility with nicking endonuclease such as N.BstNBI, such method would be also appropriate to perform cycles of concatenation according to the embodiment based on phosphorothioate nucleotides (see above): indeed, the material for each cycle of concatenation can be generated by amplification via polymerase chain reaction using biotinylated oligonucleotide primers (encompassing the recognition sequence of a restriction endonuclease) and nucleotide triphosphorothioates that assemble to form the non-digestible strands. The use of normal nucleotide triphosphates in the oligonucleotide primers ensures that only 5′-overhangs are created upon digestion of the heteroduplex DNA with the restriction endonuclease.

In another embodiment, partitioning of each target nucleic acid sequences of a starting repertoire can be performed in aqueous compartments of water-oil emulsions (Tawfik & Griffiths, 1998). A cycle of concatenation can be achieved provided that the three steps of a cycle of concatenation (nicking, elongation, ligation) are performed sequentially in the water-oil emulsions prior disruption of the water-oil emulsions for cloning or amplification of the concatenated repertoire by polymerase chain reaction.

Filling-In by Polymerase

The second step of a cycle of concatenation involves second-strand DNA synthesis by incubating the nicked DNA repertoire with a nucleic acid polymerase and nucleotide triphosphates. In a preferred embodiment, the nucleic acid polymerase should be a DNA polymerase with strand displacement activity at low temperature rather than a thermophilic DNA polymerase. DNA polymerase with strand displacement activity at low temperature such as Klenow Fragment DNA polymerase I (at 37° C.), Klenow Fragment DNA (3′-5− exo⁻) polymerase I (at 37° C.), and phage Phi29 DNA polymerase are therefore most appropriate for the elongation step. Addition of single-strand DNA-binding protein such as E. coli SSB or T4 gene 32 protein also facilitates unpairing of the cohesive-ends, thereby allowing the polymerase to perform more efficiently the elongation step, thus increasing the yield of blunt-ended linearised product. Alternatively, unpairing of long cohesive ends can be achieved by raising the temperature of the solution. However, if the starting repertoire is comprised within a collection of replicons, the use of elevated temperatures for strand dissociation followed by second-strand DNA synthesis with a thermophilic DNA polymerase would favour strand exchange and therefore shuffling between copies of different target nucleic acid sequences upon ligation. Nevertheless, provided that the nicked DNA repertoire has not been completely denatured prior to elongation (e.g. by incubation at temperatures above 80° C., or alkaline treatment), it is possible to achieve conversion of the nicked DNA repertoire into blunt-ended DNA molecules by incubation with thermophilic DNA polymerases such as Vent DNA polymerase, Vent (exo⁻) DNA polymerase, or Bst DNA polymerase (large fragment) at temperature ranging between 55° C. and 70° C.

Ligation

The third step of a cycle of concatenation involves blunt-end ligation of the two copies of each target nucleic acid sequences of a repertoire using a nucleic acid ligase such as T4 DNA ligase or E. coli DNA ligase. The resulting ligated material, i.e. the concatenated repertoire, comprises a collection of nucleic acid molecules wherein not more than two copies of each target nucleic acid sequences are linked together in head-to-tail orientation on the same DNA molecule. It can either be directly used as the substrate for a new cycle of concatenation, or used for transformation of a cell host such as a bacterial cell for propagation, or used for amplification by polymerase chain reaction with suitable oligonucleotide primers which encompass the target nucleic acid sequences. After each cycle of concatenation, the target nucleic acid sequence is at most duplicated. Thus, by repeatedly using the concatenated repertoire obtained after a cycle of concatenation as new starting repertoire for a next cycle of concatenation, the number of concatenated copies of each of the target nucleic acid sequences present in the first starting repertoire will increase according to the formula 2ⁿ wherein n is the number of concatenation cycles (e.g. after four cycles, 16 head-to-tail concatenated sequences are assembled in head-to-tail orientation on the same DNA molecule).

In a number of cytokines such as the TNF-family, the active form of the cytokine is a trimer. The present invention provides also for the creation of a concatenated repertoire of trimeric (and multimers thereof) target nucleic acid sequences. For example, such trimeric repertoire could be created from the concatenated repertoire obtained after a first cycle of concatenation. In a second cycle, this repertoire would be treated such that only one copy of each concatenated nucleic acid sequence is targeted for concatenation The product of the second cycle of concatenation would then mainly comprise a collection of nucleic acid molecules wherein not more than three copies of each target nucleic acid sequences are linked together in head-to-tail orientation on the same DNA molecule. In the second cycle, targeting the concatenation to only copy of each concatenated nucleic acid sequence would be achieved by using a different nicking endonuclease than that used for the first cycle of concatenation. To that end, the recognition sites would therefore be oriented in opposite direction (3′-side versus 3′-side), at both ends of first copy of the concatenated nucleic acid sequence. The 3′-end recognition site, e.g. the recognition site at the junction between the first and second copies of the concatenated nucleic acid sequence, could be specifically engineered upon the first concatenation cycle (i.e. in a process similar to the creation of an SpeI site as described in examples 1 and 2 in the next section). Another approach aiming at creating concatenated repertoire of trimeric (and multimers thereof) target nucleic acid sequences would entail two cycles of duplication of target nucleic acid sequences (thus creating tetrameric sequences), followed by at least partial removal of one copy of target nucleic acid sequence from the resulting tetramer. This could be achieved in a semi-controlled fashion by cutting the concatenated nucleic acid sequences at the 3′-end (or 5′-end), and then digesting the DNA with a nuclease, before religating the ends. Another method allowing controlled deletion of the fourth copy of target nucleic acid sequence would consist in cutting the concatenated nucleic acid sequences at the 3′-end (or 5′-end), and then ligating a double-stranded DNA linker carrying a type IIs restriction site such as BsgI or BpmI (which cut DNA at a 14-16 nucleotide distance of the 3′-end of the cognate restriction site). By incubating the DNA with the restriction endonuclease, a DNA fragment of 14-16 nucleotide length is therefore removed. By repeating this procedure (annealing and cutting), the fourth copy of target nucleic acid sequences can be progressively and entirely removed since the restriction pattern of the type IIs restriction enzymes is known.

Screening

The present invention provides for creating a repertoire of concatenated nucleic acid sequences such that each of these sequences results from the concatenation of a single template sequence from the repertoire. Therefore, by cloning the concatenated repertoire into an expression system, a repertoire of concatenated polypeptides corresponding to the translation of the concatenated nucleic acid sequences can be obtained. Such repertoire can be screened for concatenated polypeptides exhibiting function and/or binding properties. Whilst such expression systems can be used to screening up to 10⁶ different members of a repertoire, they are not really suited to screening of larger numbers (greater than 10⁶ members). Of particular use in the construction of large repertoire of the invention are selection display systems, which enable the production of encoded concatenated polypeptides. Encoded polypeptide members of a repertoire can be selected or screened for binding or function and further characterised by recovery of the corresponding nucleic acid sequences. Any selection display system (such as filamentous phage, bacteriophage lambda, T7 bacteriophage, E. coli display, yeast display, peptide-on-plasmids or ribosome display) may be used in conjunction with a concatenated repertoire according to the invention. Methods for construction and selection for isolating desired members of large repertoires are known in the art.

In concatenated polypeptides, the one-dimensional tandemly-repeated polypeptide sequence can upon folding generate higher-order molecules with symmetries. For example, a polypeptide comprising eight repeats of an α/β supersecondary unit may fold into a TIM-like barrel structure with an axis of twofold rotational symmetry pointing into the pocket of the barrel. Thus, symmetry can be generated through packing of supersecondary units in an ordered ensemble. Although all the units are linked together via the polypeptide backbone, it is the mesh of non-covalent contacts (comprising Van der Waals and hydrophobic interactions, hydrogen bonds and salt bridges) which creates the symmetrical assembly. Such assembly can be further stabilised by covalent bonding via cystine residues. For example, heavy-chains of immunoglobulins form covalent homodimers via disulphide bonds in the hinge region. As a result, the pseudo two-fold rotational axis of symmetry is forced to pass perpendicularly through the disulphide bonds. The concatenated polypeptide repertoires described in this invention are particularly appropriate to exploit the use of cystines as centres for rotational axis of symmetry. Indeed, since each concatenation cycle results in the generation of not more than two copies of a target nucleic acid sequence, most concatenated polypeptides will automatically contain an even number of cysteine residues which can pair to form disulphide bonds. In other words, the potential usefulness of such concatenated polypeptide repertoires can be illustrated by the 20-fold increased likelihood that two cysteine residues will be present at given positions in a duplicated peptide versus a randomised peptide of the same length.

Uses and Advantages of the Invention

The present invention may be used to give rise to at least two different types of polypeptide repertoire. The first type of repertoires, symmetry-based protein repertoires, are useful for the de novo design of new proteins based either on existing architecture, or completely new folds. The possibility to create entirely novel proteins with tailored receptor, sensory and catalytic functions depends on such an ability to build novel proteins. There is plentiful evidence of the evolutionary role of protein domains (defined as entities or building blocks that are present in single or multidomain proteins, and are thought to form stable collapsed folding units, which may eventually interact to assemble) for the generation of protein diversity (for example by duplication, swapping, transposition and recombination). Statistical and structural analysis have revealed that the average chain length of protein domains ranges between 100 and 150 amino-acid residues (Savageau, 1986; Berman et al., 1994; Xu & Nussinov, 1997).

The use of symmetry (by the means of concatenated polypeptides) easily allows the attainment of a chain length suitable for protein domains while keeping the combinatorial sequence space within experimental size. One successful example is given by Schafmeister et al. (1997) who designed a 4-helix bundle of 108 residues, comprising a reduced alphabet of seven amino acids and identical helices. Biophysical analyses of the recombinant protein revealed a monomeric state and buried amide bonds with protection factors similar to that of known proteins. By the rules of symmetry in 4-helix bundles, a residue which is suitable (or not suitable) for core packing of one helix is most likely to be suitable (or not suitable) at the same position in the other helices. If this position were to be randomly mutated in each helix in a combinatorial approach, only one clone in 1.6×10⁵ molecules would have the appropriate symmetrical solution. On the other hand, the likelihood of finding the right solution increases by 8×10³-fold in a tandemly-repeated repertoire. Thus, it is expected that proteins displaying biophysical parameters akin to natural proteins should be recovered at higher frequencies from symmetry-based protein repertoires, than from purely combinatorial protein repertoires.

In line with this, 80-residue polypeptides recovered from a combinatorial repertoire using Glu, Leu and Arg as minimal alphabet, did not exhibit slowly exchanging amide hydrogens which would have suggested the existence of an hydrophobic core (although they displayed some degree of helical content and co-operative thermal unfolding which would suggest coiled-coil structures; Davidson et al., 1995). The symmetrical orientation of amino acid residues might also facilitate the design of metal-sensing in a symmetry-based protein, due to the geometrical positioning of electron pairs required for metal-co-ordination. Finally, duplication of protein domains might also trigger the design of new proteins via domain swapping (Heringa & Taylor, 1997).

The second type of repertoires, tandemly-repeated peptide repertoires, are useful for generating concatenated peptides of extended structures like beads-on-string with biophysical properties (such as extended 3D-structure and adhesion) for example similar to that of silk, fibroin, elastin, collagen or keratin, or to generate specific ligands for proteins exhibiting rotational symmetries (for example receptors, viral envelopes, enzymes, DNA and metal surfaces).

Some cell receptors exhibit a two-fold rotational symmetry at their ligand-binding site: as a result, the binding site is prone to bind to molecules which also exhibit a two-fold rotational symmetry. For example, Tian et al. (1998) have isolated a small non-peptidic molecule with two-fold rotational symmetry, that binds to the granulocyte-colony stimulating factor (G-CSF) receptor.

In another example, Wrighton et al. have isolated a 20-residue cyclic peptide by phage display, which functions as a symmetrical homodimer to trigger dimerisation of the human erythropoietin receptor (EPOR) (Livnah et al., 1996). It should be noted that the starting peptide repertoire was not designed to encode tandemly-repeated peptides: the use of high-valency for phage display (using pVIII as carrier) and the mode of receptor presentation for selection may have put selective pressure for symmetrical peptide dimers on phage. Such self-association of peptides has also been observed by Wang & Pabo (1999) upon selection of peptide repertoires fused to the N terminus of Zif12 attached to pIII, on a bait DNA fragment.

Furthermore, Zwick et al. (2000) have observed that peptides containing one (or two) cysteine residue can form covalently-bound homodimers when displayed as fusions with pVIII on phage. Thus, at least in some circumstances, it would appear that peptide dimerisation can occur on the phage surface. This is nevertheless conceptually different to the concatenation approach described in this patent application: (1) in the present approach, the polypeptides are covalently linked in a head-to-tail orientation whereas self-association of peptides on phage involves a free N-terminus for each peptide; (2) the present approach affords to generate polypeptide multimers whereas self-association of peptides on phage would appear to be limited to homodimeric structures; (3) in the present approach, each unit of a polypeptide multimer is encoded by its own genetic information, whereas a unique DNA fragment encodes both copies of self-associated peptides on phage.

This is particularly relevant for further improvement of concatenated polypeptides by either error-prone PCR or DNA shuffling, in a process which mimics more closely the natural evolution of protein diversity. Finally another advantage of concatenated peptide repertoires is the potential for even greater sequence space diversity due to permutations. Indeed, supposing a repertoire of trimeric pentapeptides (i.e. in the form of (ABCDE)₃), each 15-residue peptide also encompasses all duplicated, permutated forms of the basic motif: (BCDEA)₂, (CDEAB)₂, (DEABC)₂, (EABCD)₂, thereby multiplying the sequence space of the repertoire by (n−1)-times (wherein n is the number of amino acid residue in the basic peptide motif).

Natural sequences (such as cDNAs and genomic DNA) are an important source of diversity for libraries of concatenated polypeptides. Repeats are widespread in natural proteins; a general algorithm for repeat detection found that 14% of proteins contain duplicated sequence segments and identified a number of previously unknown repeat families (Marcotte 1998); thus it seems that many natural sequences can provide useful features when concatenated. Since natural sequences already encode secondary structural elements, constructing new protein domains by organising these in new ways is more likely to result in proper folding than starting from random sequence. It has been shown that novel folded protein domains can be isolated by combinatorial shuffling of short natural sequences (Riechmann & Winter 2000). Concatenation provides another avenue for rearranging natural sequences that may yield folded domains at relatively high frequencies and be advantageous for functions benefiting from symmetry, such as binding to multimeric receptors and allostery; or simply by providing a higher local concentration of binding sites or enzyme active sites. For human therapy, concatenated proteins engineered from natural human sequences have the important advantage of containing no or very few non-human peptides, and can be much less immunogenic.

The invention is further described, for the purpose of illustration and not by way of limitation, in the following experimental section.

### EXPERIMENTAL

The present examples describe the implementation of the method to create concatenated DNA sequences, its use to create repertoires of encoded concatenated polypeptides, and the selection/characterisation of a 30-mer polypeptide resulting from the duplication of a 15-mer peptide, which binds as an antagonist to the soluble domain of the human erythropoietin receptor.

### Example 1

### One Cycle of Concatenation on a Randomised 84-bp DNA Sequence

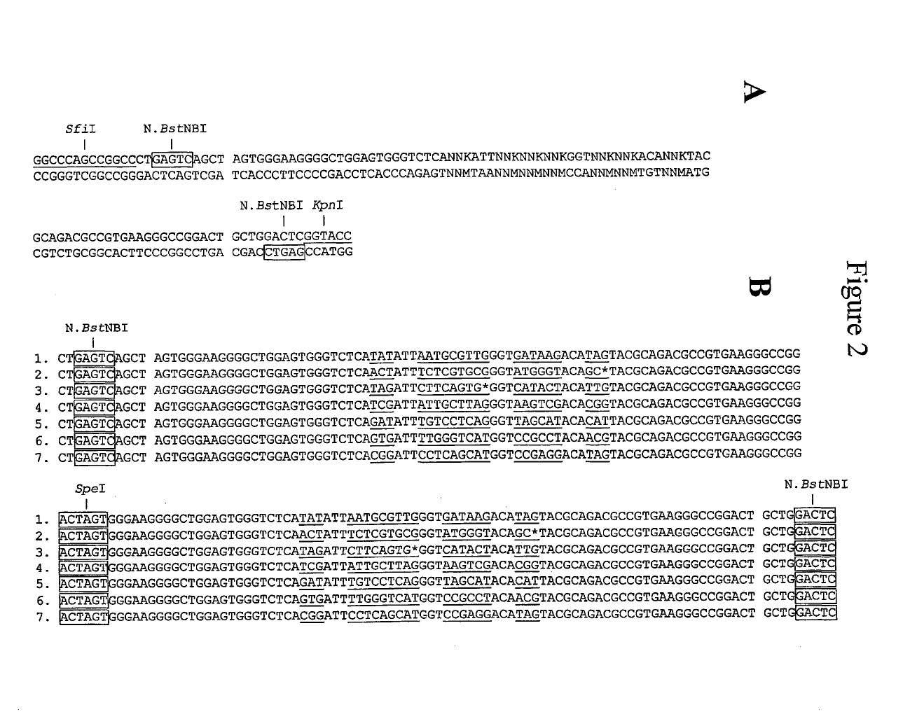

By PCR, two N.BstNBI sites (one at each end and in opposite orientation) were appended to a 84-bp DNA sequence encompassing the randomised V_{H}-CDR2 of a synthetic repertoire of human ScFV, pIT2-I repertoire (Tomlinson et al.), which was subsequently cloned into pK4, a phagemid vector devoid of N.BstNBI sites (repertoire size: ˜10³ clones) (FIG. 2.a). To perform a cycle of concatenation, a three-step approach was followed:

Incubation of pK4-V_{H}-CDR2 with N.BstNBI to create a single-stranded DNA nick at the 5′-end of each DNA strand of the 78-bp target sequence. Agarose gel analysis of the “nicked” DNA confirmed a change in electrophoretic mobility when compared to untreated DNA.

The second step aims at filling the 5′-overhangs with nucleotides in presence of a DNA polymerase. Best results were obtained with Klenow Fragment of DNA polymerase I (at 37° C.) but other polymerases exhibiting strand-displacement activity such as Vent DNA polymerase and Vent (exo⁻) DNA polymerase also gave satisfactory results (at 55° C.). The product of this reaction is thus a linearised plasmid DNA with two copies of the 84-bp target sequence, one at each at ends and oriented in the same direction, as confirmed by agarose gel analysis.

In the third step, the blunt ends are ligated with each other, thereby linking the two copies of the 84-bp target sequence in head-to-tail orientation. To improve self-ligation of the linearised vector, the DNA concentration was lowered to ≦5 μg/ml. Indeed, ligation of blunt-ended DNA at higher concentration may favour intermolecular reactions instead of intramolecular ones, that would eventually result in shuffling of copies derived from different target nucleic acid sequences.

Following transformation, several clones were analysed by PCR screening to assess the length of the target DNA sequence located between the N.BstNBI sites. As expected, most of them were in accordance with an expected length of 168-bp (28 out of 32 clones, 87.5%). A restriction test was also performed to assess the correctness of the junction between the two 84-bp copies. Thus, the 84-bp target sequence was constructed with the second half of the SpeI restriction site at the 5′-end (AGT) and the first half of the same restriction site at the 3′-end (ACT). After a cycle of concatenation, the SpeI restriction site (5′-ACTAGT-3′) is reconstituted at the junction between the two 84-bp copies. As expected, SpeI digestion of all plasmids containing a duplicated 84-bp fragment revealed the presence of a unique SpeI site which was not present in the parental plasmid. Finally, the target nuclei acid sequences of seven positive transformants were sequenced: all clones encoded exact duplications FIG. 2.b). Moreover, since V_{H}-CDR2 was cloned as a randomised repertoire into pK4, DNA sequencing also revealed that the copies of each concatenated target nucleic acid sequences were identical. Thus, this example demonstrates that the present method is suitable (1) for concatenating target DNA sequences (up to 84-bp), and (2) for self-concatenating target DNA sequences in a repertoire of average complexity (10³ different units).

### Example 2

### Four Cycles of Concatenation on a Randomised 84-bp DNA Sequence

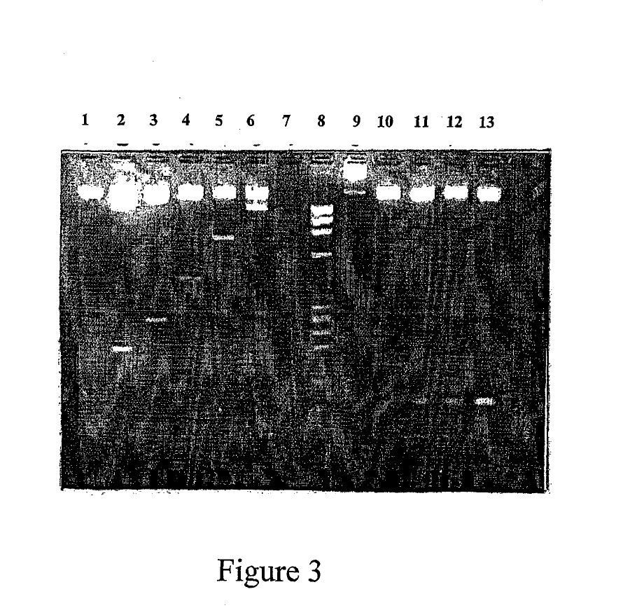

The above described method can be repeated several times on the same DNA template. Indeed, after a first cycle of duplication, the N.BstNBI sites are not destroyed, no additional N.BstNBI sites have been created, and the concatenated DNA sequences are still comprised within the pK4 plasmid. This opens the possibility to further concatenate target nucleic acid sequences by performing several sequential cycles of concatenation (FIG. 3).

Thus, double-stranded DNA was prepared from the pooled transformants obtained after the first cycle of duplication (see Example 1) and a cycle of concatenation was performed as described in Example 1. PCR screening of 28 transformants revealed 23 clones (82%) carrying a concatenated DNA sequence of expected length (4×84-bp=336 bp). SpeI digestion of all positive clones confirmed the presence of three SpeI sites (two resulting from the duplication of the SpeI site created after the first cycle of concatenation, and one created by the ligation of the concatenated 168-bp sequences) in most clones (19 out of 22 clones, 86%).

To obtain eight concatenated target nucleic acid sequences per clone (8×84 bp=672 bp), a third cycle of concatenation (as described in Example 1) was then performed using the DNA prepared from pooled transformants obtained after the second cycle of concatenation. PCR screening on 20 transformants revealed that 13 out of 20 clones (65%) carried the insert of expected length. SpeI digestion of ten of these positive clones confirmed the presence of seven SpeI sites (four resulting from the duplication of the SpeI site created after the first cycle of concatenation, two resulting from the duplication of the SpeI site created after the second cycle of concatenation and one created by the ligation of the concatenated 336-bp sequences) in most clones (9 out of 10 clones, 90%).

Finally, using a single clone carrying eight copies of the starting target nucleic acid sequence as source of double-stranded DNA, a fourth cycle of concatenation (as described in example 1) was performed to generate sixteen copies of the starting target nucleic acid sequence per transformants (16×84 bp=1344 bp). PCR screening on several transformants revealed that they carried the insert of expected length. SpeI digestion of two of these positive clones confirmed the presence of 15 SpeI sites (eight resulting from the duplication of the SpeI site created after the first cycle of concatenation, four resulting from the duplication of the SpeI site created after the second cycle of concatenation, two resulting from the duplication of the SpeI site created after the third cycle of concatenation and one created by the ligation of the concatenated 672-bp fragments).

This example demonstrates that (1) target nucleic acid sequences up to 672-bp can be concatenated using the protocol described in Example 1, and (2) target nucleic acid sequences up to 84-bp can be submitted to multiple cycles of concatenation (up to four times) to create concatenated DNA sequences of 1.3 kbp length that could potentially encode for 448-residue proteins (about 50 kD) comprising 16 tandemly-repeated copies of a 28-residue polypeptide.

### Example 3

### Construction of Encoded Concatenated Polypeptide Repertoires

The potential of the present invention was further evaluated by constructing encoded concatenated polypeptide repertoires of much larger complexities (≧10⁷ individual clones). First, randomised 6- and 15-residue peptide repertoires were constructed by cloning a 18-bp randomised DNA fragment and a 45-bp randomised DNA fragment (using NNK codons) into pK10-AmbS and pK10-2AmbS. The synthetic DNA fragments were designed such that the 18-bp and the 45-bp target DNA sequences were encompassed by two N.BstNBI nicking sites in opposite directions. Both sites were positioned such that (1) upon nicking, the 5′-overhangs would only comprise the target DNA sequence and (2) the nicking sites are located at the junction between coding triplets. The repertoires (herein named pK10-1×6-mer and pK10-×15-mer) of greater than 5×10⁸ ampicillin-resistant clones were obtained in E. coli TG1 cells. Greater than 90% of the clones had a correct size insert as demonstrated by PCR screening (n=30), and automated DNA sequencing of 23 clones revealed correct nucleotides sequences for all (no deletions, nor insertions), with no nucleotide bias in the randomised sequences.

Next, double-stranded DNA was prepared from the pooled transformants of each repertoires, and submitted to a cycle of concatenation as described in Example 1 but adapted for larger DNA samples. The resulting concatenated repertoires (herein named pK10-2×6-mer, and pK10-2×15-mer) each comprised at least 2×10⁸ individual clones (by combining pK10-AmbS and pK10-2AmbS transformants), wherein greater than 80% of the clones had a correct size insert as demonstrated by PCR screening (n=40). Automated DNA sequencing revealed that 13 out 14 positive clones from the 2×6-mer concatenated encoded repertoire carry exact tandem repeats of the 18-bp randomised target DNA sequence. The eighth clone contains a mixed (or shuffled) concatenated DNA sequence (i.e. two different 18-bp DNA sequences linked together in head-to-tail orientation). A similar result was obtained with the 2×15-mer encoded repertoire: 7 clones out of 9 had a correctly duplicated 45-bp DNA sequences while the remaining two clones had different 45-bp DNA units linked together. Possibly, such shuffled concatenated sequences arise during the nicking step at 55° C. which may favour strand exchange between two plasmid, or, more likely, by spurious ligation of two or more linearised vectors (each carrying a different target nucleic acid sequence) followed by resolution in bacteria.

In line with this, we have observed an even greater frequency of shuffled concatenated sequences after the second cycle of concatenation because the 4×6-mer and the 4×15-mer concatenated repertoires were assembled via an intermediate PCR amplification step. Thus, after the second cycle of concatenation, the linearised DNA was ligated and directly submitted to PCR amplification of the concatenated DNA inserts. After digestion, the concatenated DNA sequences were then cloned into a phage vector, Fd-tet-SN, yielding a repertoire of 8.35 10⁷ clones for the 4×6-mer repertoire (herein named Fd-4×6-mer), and 1.35×10⁸ clones for the 4×15-mer repertoire (herein named Fd-4×15-mer). For the Fd-4×6-mer repertoire, PCR screening (n=12) revealed that 60% of the clones had a correct 72-bp DNA insert, of which 30% of the DNA sequences were exact repeats, thereby yielding a desired repertoire size of 1.4×10⁷ clones. For the Fd-4×15-mer repertoire, PCR screening (n=30) revealed that 47% of the clones had a correct 180-bp DNA insert, of which 57% of the DNA sequences were exact repeats, thereby yielding a desired repertoire size of 3.6×10⁷ clones. Among the clones with correct size DNA insert, the percentage of shuffled inserts were thus higher, with combinations of target DNA sequences such as AABB, ABCC or AABC (with A, B, and C as different 18-bp or 45-bp target nucleic acid sequences from the starting repertoires). Finally, the number of 6-mer and 15-mer motifs containing at least one stop codon (TAG) corresponds with the predictions, according to the statistical rule P=1−(31/32)^{N} where N is the number of amino acids per peptide: 17% and 33% for the 6- and 15-mer, respectively.

Finally, for the purpose of biopanning (see Examples 4 and 5), the repertoires of 1×6-mer, 2×6-mer, 1×15-mer and 2×15-mer were also subcloned from their starting vectors, pK10-AmbS and pK10-2AmbS, into the Fd-Tet-SN phage vector. The quality of these libraries was also assessed by PCR screening and DNA sequencing of several clones:

Fd-1×6-mer: 100% of the clones with 18 bp inserts (12/12), all DNA sequences were correct (4/4), repertoire size: 1.26×10⁸ clones.

Fd-2×6-mer: 83% of the clones with 36 bp inserts (10/12), of these, 85% DNA sequences are exact repeats (6/7), repertoire size: 6.19×10⁷ clones.

Fd-1×15-mer: 100% of the clones with 45 bp inserts (15/15), all DNA sequences were correct (6/6), repertoire size: 1.05×10⁸ clones.

Fd-2×15-mer: 53% of the clones with 90 bp inserts (8/15), of these, 85% DNA sequences are exact repeats (⅞), repertoire size: 5.8×10⁷ clones.

In conclusion, we have demonstrated that even in the context of large DNA diversity, the present invention is suitable to generate repertoires of encoded concatenated polypeptides (carrying not more than two, or four copies of a basic polypeptide element) wherein a large fraction of these results from the concatenation of a single element of the repertoire.

### Example 4

### Concatenation of a Repertoire of Random Genomic Segments

The source of sequence diversity for concatenated polypeptide repertoires of the present invention may be completely synthetic or may be derived from naturally occurring sequences. To demonstrate the latter, a repertoire of sequences was constructed from E. coli genomic DNA, using tagged random primer amplification. The tag or constant part of the random primer encoded a N.BstNBI nicking site and BstXI restriction site, and size-selected fragments were ligated into the BstXI sites of pW564 (a derivative of pK10-AmbS that encodes barnase as a translational fusion 5′ of the cloning site). The nicking sites were positioned such that inserts that were in-frame with both barnase and coat protein III (pIII) would remain in-frame after duplication. The insert size was chosen to represent ˜50 amino acids, unlikely to be large enough to form a folded domain on its own but to be the size of a typical folded domain (˜100 amino acids) when duplicated. This starting library, named library#4, comprised 1.4×10⁹ transformants.

Because of the random nature of the inserts, ˜90% of clones were out-of-frame or contained stop codons. These were eliminated using a trypsin-sensitive helper phage (Kristensen & Winter, 1998). After treatment with trypsin, phage from clones with inserts that are out-of-frame with pIII are no longer infectious; but phage from clones in-frame with pIII are still infectious by virtue of their trypsin-resistant pIII. DNA sequencing showed that 16 of 18 clones (89%) were in-frame and without stop codons; all inserts could be identified as segments from the E. coli genome, and encoded peptides of 37-63 amino acids.

Plasmid DNA was prepared and submitted to one cycle of concatenation by nicking, extension, and re-ligation in the manner of Example 1; the linear product of the extension reaction was gel-purified before re-ligation to increase the proportion of concatenated clones. The resulting repertoire, library#8, comprised 6.0×10⁹ transformants. DNA sequencing of eight clones showed that all were precise duplications of E. coli genomic segments, and encoded peptides of 92-132 amino acids.

Phage were prepared from library#8 and subjected to proteolytic selection in the manner of Riechmann & Winter (2000). Phage were treated with trypsin and thermolysin and then allowed to bind to biotinylated barstar immobilised on streptavidin-coated wells. Phage that remain intact despite protease treatment will be able to bind barstar by virtue of the barnase-insert-pIII fusion; phage in which the insert is cleaved by protease will no longer be able to bind to barstar. The number of surviving phage was 4.7×10⁻⁶ lower than the number of input phage. These were propagated in bacteria and subjected to a second round of proteolytic selection. There was a 64-fold increase in the fraction of surviving phage relative to round 1. Individual clones from round 2 were screened by phage ELISA for protease resistance. 2 of 40 clones showed protease resistance of ≧40% (ratio of ELISA signal of protease-treated phage compared to untreated phage); these two clones (8r1C2 and 8r1E3) are candidates for novel folded protein domains derived by sequence concatenation.

### Example 5

### Concatenation of Linear DNA Fragments Anchored to a Surface

In the above examples, the concatenation procedure depends on the use of circular plasmids so that, after nicking and extension, ligation at low DNA concentration favours intra-molecular ligation. It may be useful to create concatenated repertoires of linear DNAs such as PCR products. This is important for expression/selection methods that can be carried out completely in vitro; it may also be expedient when only higher levels of concatenation are required in the final repertoire (early rounds of concatenation could be done with linear DNAs and only the final round need be cloned into a plasmid vector).

If both ends of a linear DNA molecule are anchored to a surface, there should be a similar bias towards self-ligation as with circular DNAs. One way to achieve this anchoring is to generate the linear DNA using PCR wherein both primers are biotinylated at their 5′ ends; a surface coated with streptavidin can then capture both ends of the resulting PCR product. Other anchoring mechanisms could be used, so long as they do not interfere with the enzymatic reactions for concatenation or PCR, for example, covalent linkage of amino-modified DNAs or capture with site-specific DNA binding molecules (proteins, nucleic acids, or other chemicals).

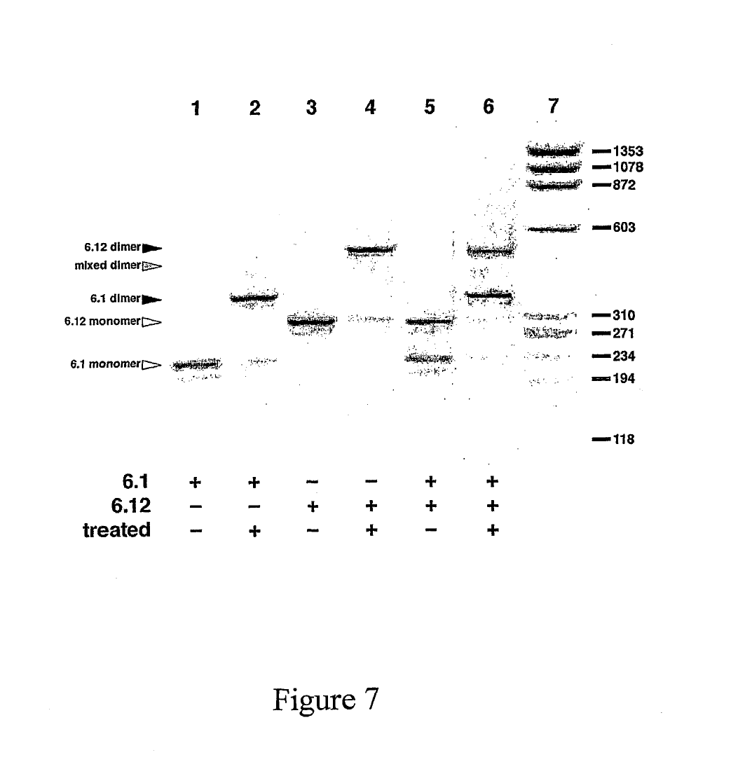

This was tested using two clones, 6.1 and 6.12, carrying 138 bp and 210 bp E. coli genomic segments flanked by N.BstNBI sites. These inserts were separately amplified by PCR using biotinylated primers. The PCR products were bound to streptavidin magnetic beads, either separately or in combination The beads were then treated with N.BstNBI, Klenow, and T4 DNA ligase, with washing between incubations. Small amounts of beads, treated or untreated, were used as templates for PCR and analysed by gel electrophoresis (FIG. 7). PCR of untreated beads gives monomer-sized bands, 226 bp or 298 bp for 6.1 or 6.12. After one cycle of concatenation on the beads, the predominant band is the duplication product (clonal dimer), 364 or 508 bp for 6.1 or 6.12; some monomer band is also present. Concatenation done on beads coated with both clones, 6.1+6.12, gives both clonal dimers as the major products; there is also some of both monomers, as well as a small amount of the mixed dimer product (436 bp). The predominance of clonal dimers over the mixed dimer demonstrates that self-ligation is favoured; if ligation were random, one would expect twice as much mixed dimer as either clonal dimer.

### Example 6

### Biopanning of the Fd-1×15-mer, Fd-2×15-mer and Fd-4×15-mer Peptide Libraries on 9E10 and EC-EpoR-Fe

To evaluate the encoded concatenated polypeptide repertoires constructed in Example 3, affinity-selections by biopanning were performed with the Fd-1×15-mer, Fd-2×15-mer, and Fd-4×15-mer repertoires and two target molecules: a mouse monoclonal antibody 9E10 (which binds to the c-myc peptide epitope, EQKLISEEDL), and a chimaeric molecule comprising the extracellular domain of the human erythropoietin receptor fused to the Fc portion of human IgG1 (EC-EpoR-Fc). The antibody-combining site of 9E10 is asymmetrical due to the heterodimeric assembly of heavy and light chains, whereas the homodimeric EC-EpoR-Fc molecule exhibits a two-fold rotational symmetry at the erythropoietin binding site.

Two rounds of biopanning on both targets were performed with each of three polypeptide repertoires. A strong enrichment factor was observed with all three peptide repertoires upon selection on 9E10: indeed, the phage recovery increased considerably after round 2 when compared to round 1 (250-, 1200, and 300-fold increase with the Fd-1×15-mer, Fd-2×15-mer and Fd-4×15-mer polypeptide repertoires, respectively). In contrast, a strong enrichment only observed with the concatenated repertoires (75- and 80-fold increase with the Fd-2×15-mer and Fd-4×15-mer polypeptide repertoires, respectively) after two rounds of selection on EC-EpoR-Fc. The specificity of these strong enrichments were further confirmed by ELISA using polyclonal phage preparations from each selection round. Thus, these results validated our assumption according to which concatenated polypeptide repertoires rather than simple polypeptide repertoires are a more likely source for specific ligands to symmetrical targets, but not necessarily for asymmetrical targets. Next, individual clones were tested by phage-ELISA with the appropriate immobilised target: more than 90% of the 2×15-mer (n=48) and 4×15-mer clones (n=48) recovered after round 2 bound to EC-EpoR-Fc. Similar results were obtained with 1×15-mer (n=24), 2×15-mer (n=24) and 4×15-mer (n=24) clones isolated after the second selection round on 9E10. By DNA sequencing, a single concatenated polypeptide motif was obtained amongst nine 2×15-mer clones that bind to EC-EpoR-FC: (PLACHGATLEQTYAL)₂. This concatenated polypeptide sequence (hereby named MP sequence or MP peptide) was also identified in positive clones recovered from the 4×15-mer repertoire (7/9) along with two clones containing a further concatenated version of the same 15-residue polypeptide, (PLACHGATLEQTYAL)₄. This concatenated polypeptide motif shares no homology with human erythropoietin, nor with the EPOR-specific peptides which were isolated by Wrighton et al. (1996) and McConnell et al. (1998). The 9E10-binding clones were predominantly cysteine-free sequences: GMSPGNHTHRFLLSE from the 1×15-mer peptide repertoire (5 out of 8 clones), (LNDVGLLSEFMAFDR)₂ from the 2×15-mer peptide repertoire (5 out of 8 clones), (IAKQDTRAQMLVSEE)₄ from the 4×15-mer peptide repertoire (5 out of 8 clones), wherein a sequence motif that closely match to the c-myc epitope could easily be mapped (underlined).

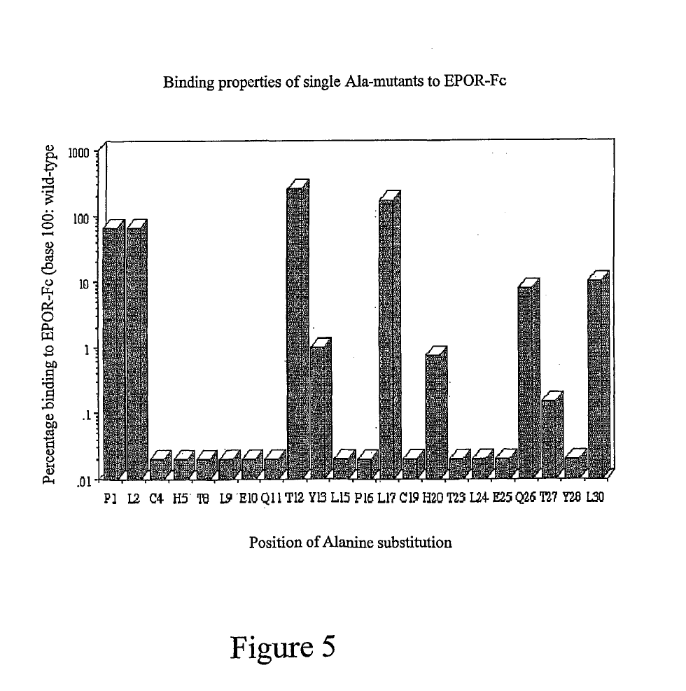

Next, the MP sequence was characterised by alanine-scanning mutagenesis, affinity measurements and stoichiometric analysis. Alanine-scanning mutagenesis was meant to delineate the MP residues that contribute to EC-EpoR-Fc binding. In total, 22 residues (all amino acids except Gly an Ala) were individually replaced by alanine and the binding activity of these phage-displayed mutants was investigated by phage-ELISA on immobilised EC-EpoR-Fc. The results are listed in FIG. 5. Mutation of each of the cysteine residues (C₄ and C₁₉) had a strongly disruptive effect on EC-EpoR-Fc binding, thereby suggesting that these residues react with each other to form a 16-residue cycle. In line with this, the EC-EpoR-Fc binding activity of MP-bearing phage was totally abolished upon reduction/alkylation of the phage prior ELISA (data not shown). Five other pairs of residues showed similar behaviour upon individual alanine substitution: strongly disruptive effect for H₅ and H₂₀, T8 and T₂₃, L₉ and L₂₄, E₁₀ and E₂₅, or no detectable effect for L₂ and L₁₇. These symmetrical effects are not found within other pairs of residues: the largest discrepancies (by more than 1000-fold) are observed between P₁ and P₁₆, Q₁₁ and Q₂₆, T₁₂ and T₂₇, and L₁₅ and L₃₀, whereas the Y₁₃/Y₂₈ pair is ten-fold less asymmetrical. Overall, these results indicate that (1) 80% of the mutated residues induce a ≧100-fold reduction in MP binding activity, and (2) these critical residues span most of the peptide length (from position 4 to position 28) but not the border positions. Along with the crucial role of the cysteine residues, these results suggest that MP may form a compact structure upon binding to one molecule of EC-EpoR-Fc, instead of an extended polypeptide with two independent EC-EpoR-Fc-binding sites, such as those selected in the 9E10-binding peptides. Furthermore, the results suggest that MP binding to EC-EpoR-Fc might exploit some elements of two-fold rotational symmetry. As shown in FIG. 5, the hot-spots of binding energy to EC-EpoR-Fc, H₅-T₈-L₉-E₁₀ and H₂₀-T23-L₂₄-E₂₅ are reflecting towards each other through a rotational axis of symmetry centred on the disulphide bond. Putatively, these regions might be directly involved in similar intermolecular contacts with each chain of EC-EpoR-Fc. In line with this, we have also observed by ELISA that the E1 peptide does not bind to immobilised monomeric sEPOR-Fc (obtained by site-directed mutagenesis of the two Cys residues within the human IgG1 hinge) when it is fused to the maltose-binding protein (MP-MBP).

Moreover, the apparent dissociation constant (K_{d}) of MP-MBP for EC-EpoR-Fc as determined by SPR on the BIAcore instrument is almost identical whether the MP-MBP (0.72±0.15 μM) or EC-EpoR-Fe (0.63±0.17 μM) is first immobilised on the streptavidin-coated Sensorchip. Conversely, it is therefore not really surprising that the symmetry breaks out in the second half of the 16-residue cycle (from T₁₂ to P₁₆) because the symmetrically-related counterpart (from T₂₇ to P₁) via the disulphide bond is disrupted between L₃₀ and P₁.

Without a crystal structure of the MP/Ec-EpoR-Fc complex, it is however unclear whether the contribution of an MP residue in these parts to EC-EpoR-Fc binding is related to direct peptide-receptor interaction or to intra-molecular interactions. In an attempt to delineate the MP peptide binding site on EC-EpoR-Fe, a competition ELISA using a synthetic truncated version of the MP peptide (trMP, from A₃ to Y₂₈) was performed. While trMP peptide blocked MP phage-peptide binding to immobilised EC-EpoR-Fc with an IC₅₀ of 10 μM, no reduction in signal was observed when phage-displaying the EMP1 peptide (Wrighton et al., 1996; Johnson et al., 1998) were incubated with trMP concentration up to 1 mM (data not shown). Similar results were observed in a reverse assay wherein MBP-fusions of the MP and EMP1 peptides were immobilised and incubated with 0.1 μM EC-EpoR-Fc and increasing amounts of trMP peptide (from 0.1 μM up to 1 mM) (data not shown). Taking into account that EMP1 binds to the active site of EpoR as a homodimer with a reported IC₅₀ of 0.2 to 5.0 μM (Wrighton et al., 1996; Johnson et al., 1998), our results indicate that the selected MP peptide recognises another epitope on EC-EpoR-Fc yet to be identified. However, the binding site does not involve the Fe portion of the molecule since no binding was observed when phage displaying the MP peptide were incubated in wells coated with a recombinant IgG1 Fe molecule (a covalent 50 kD homo-dimer comprising the hinge and domains CH₂ and CH₃). A similar result (i.e. binding outside the biological ligand-binding site) has been reported for the symmetrical molecule SB-247464 which recognises the homodimeric G-CSF receptor (Tian et al., 1998). Large and complex proteins with modular architecture indeed appear to have multiple ligand-binding sites located at the slits between symmetrical modules.

### Example 7

### Biopanning of the Fd-1×6-mer, Fd-2×6-mer, Fd-4×6-mer, Fd-1×15-mer Fd-2×15-mer and Fd-4×15-mer Peptide Libraries on Ferritin

Horse spleen ferritin is a 660 kD oligomeric molecule comprising 24 four-helix bundles arranged in 432 symmetry to form a spherical shell around a mineral core of ferrihydrite (composed of ˜2000 Fe atoms). After two rounds of selection, phage-ELISA on immobilised ferritin revealed specific enrichment from the 1×15-mer library (40 positive clones out of 40), the 2×15-mer library (8/40), and the 4×6-mer library (13/40). DNA sequencing of several positive clones revealed that a single peptide sequence had been selected in each biopanning, respectively: LLPRWSCGPFSCTVN (F1), (LLPRWSCGPFSCTVN)₂ (F2), and (HKPRKE)₄ (F6). Peptides F1 and F2 share the same 15-mer peptide unit which bears no similarity with the F6 sequence.

As observed with the 9E10-binding peptides (see Example 6), the ELISA signals of phage-displayed F1 and F2 on immobilised ferritin are very similar (data not shown), thereby indicating that the separate units in F2 can not effectively cooperate for binding to ferritin. This probably hints to an inadequacy between the distance separating the critical residues on F2 peptide and the distance between the binding epitopes on the highly symmetrical ferritin surface, but by further concatenating the F2 peptide, avidity effect might ultimately become again apparent. In line with this, SPR analysis of ferritin binding to immobilised maltose-binding proteins fused with F1 and concatenated derivatives has shown that the apparent dissociation constant remains constant up to 3 concatenated copies of F1 (˜2.5 μM) and then drops to 600 nM with 4×F1.

In contrast, the results from the Fd-1×6-mer, Fd-2×6-mer, and Fd-4×6-mer peptide libraries indicate that concatenation is crucial to reach the selection threshold for binding to ferritin. A single or double HKPRKE hexapeptide unit fused to maltose-binding protein displays no detectable affinity for ferritin in ELISA. The affinity is barely detectable for the triple HKPRKE hexapeptide unit (K_{d}˜20 μM, and then subsequently concatenation (n-unit of HKPRKE motif) brings the apparent dissociation constant down to subnanomolar range: K_{d}˜500 nM when n is 4 (F6 peptide), K_{d}˜8 nM when n is 8, K_{d}˜0.7 nM when n is 16. The presence of 4 positively charged residues per single HKPRKE motif hints at a major force, namely electrostatic interaction, which would stabilise the F6:ferritin complex. Indeed, the outer molecular surface of ferritin exhibits a strong negative potential (Douglas & Ripoll, 1998), and polycationic polymers (such as poly-L-lysine) have been reported to mediate ferritin adsorption to cell surface anionic sites (Skutelsky & Bayer, 1987). To confirm this hypothesis, we constructed several F6 variants wherein either the inter-motif distance was gradually increased with 1, 2 or 3 Gly residues, or wherein the F6 sequence was scrambled. By competition ELISA, we observed that these peptide constructs had affinities to ferritin very similar to that of peptide F6. This clearly demonstrates that ferritin binding by peptide F6 is primarily mediated by the density of positively charged residues rather than by weaker forces (hydrogen bonds, hydrophobic effects, . . . ) which rely more on appropriate positioning of atoms on the surface of ferritin. This result also highlights high information content of concatenated peptide libraries. In a randomised polypeptide, the likelihood that down-mutations will offset up-mutations increases with its length, so that ultimately most longer polypeptides will have average sequence profiles. In contrast, concatenation exacerbates the biophysical properties of the peptide and may increase the likelihood that the library contains peptide sequences that are marked by certain desirable functional features, such as folding initiation sites or binding sites, without the presence of irrelevant or elsewhere disruptive (e.g. with respect to binding or folding) other sequence motifs. This was clearly demonstrated upon selection of peptide F6 on ferritin wherein electrostatic interactions mediated by Arg, Lys and His residues are dominating. Indeed, the likelihood of finding a peptide containing 16 positively charged residues (at any arbitrary position) is approximately 10⁶-fold higher in a library of tetraplicated hexapeptides than in a library of filly randomised 24-mer peptides.

Methods

1. Construction of Vectors pK4, pK10, pK10-AmbS and pK10-2Ambs

Phagemid vector pK10 was constructed from Litmus 39 (New England Biolabs N3639S) using an approach combining SOE-PCR and a type IIs restriction endonuclease (BsmBI) to remove six internal N.BstNBI restriction sites and to replace the multiple cloning site in Litmus 39:

Fragment A was amplified from Litmus 39 by PCR using LJ667 and LJ668 (see Table 1) as backward and forward primers, respectively. In addition to a 5′-end extension containing a BsmBI restriction site, the resulting DNA fragment contains a silent T->C substitution at position 934 and a A->T transition at position 1255, which destroy two N.BstNBI restriction sites (one in the ampicillin resistance gene, one within the M13 origin of replication) in Litmus 39.

Fragment B was amplified from Litmus 39 by PCR using LJ669 and LJ670 (see Table 1) as backward and forward primers, respectively. In addition to a 3′-end extension containing a BsmBI restriction site, the resulting DNA fragment contains a silent T->A substitution at position 1279 of the plasmid and a C->G transition at position 1943, which destroy two N.BstNBI restriction sites (one within the M13 origin of replication, and one within the ColE1 origin of replication) in Litmus 39.

Fragment E was amplified from Litmus 39 by PCR using LJ671 and LJ675 (see Table 1) as backward and forward primers, respectively. In addition to a 5′-end extension containing a BsmBI restriction site, the resulting DNA fragment contains a C->G transition at position 1943 and a multiple cloning site for HindIII, PstI and EcoRI at the 3′-end, which destroy two N.BstNBI restriction sites (one within the ColE1 origin of replication, and one within the multiple cloning site) in Litmus 39.

1. Fragment F was amplified from Litmus 39 by PCR using LJ676 and LJ674 (see Table 1) as backward and forward primers, respectively. In addition to a 3′-end extension containing a BsmBI restriction site, the resulting DNA fragment contains the multiple cloning site for HindIII, PstI and EcoRI at the 5′-end and a silent T->C substitution at position 934, which destroy two N.BstNBI restriction sites (one within the multiple cloning site, and one within the ampicillin resistance gene) in Litmus 39.

By SOE-PCR, the purified fragments A and B on one hand, and the purified fragments E and F on another hand were linked together via their complementary ends to yield fragments AB (1.0 kb) and EF (1.7 kb), respectively. After purification, fragments AB and EF were digested with BsmBI, purified and ligated together prior to transformation of E. coli TG1 cells. The ampicillin-resistant transformants were analysed for their resistance to digestion with PleI (a restriction endonuclease which recognises the same DNA site as N.BstNBI but cuts both strands) and packaging capability as single-stranded DNA upon superinfection with M13K07 helper phage. From a positive N.BstNBI-deficient Litmus 39 clone, double-stranded DNA was prepared, digested with HindIII and EcoRI and purified.

To construct pK10, a DNA insert encoding the PelB leader sequence, a multiple cloning site and a variant of gene III devoid of N.bstNBI sites was prepared by SOE-PCR:

Fragment G was amplified from phagemid pH by PCR using LJ008 and LJ678 (see Table 1) as backward and forward primers, respectively. The resulting DNA fragment contains a HindIII restriction site at the 5′-end a silent G->A substitution which destroys the N.BstNBI restriction site at triplet encoding residue X of coat protein m.

Fragment H was amplified from phagemid pHENI by PCR using LJ679 and LJ680 (see Table 1) as backward and forward primers, respectively. In addition to a 3′-end extension containing an EcoRI restriction site, the resulting DNA fragment contains two silent G->A substitutions which destroy the N.BstNBI restriction sites at triplets encoding residues X and X of coat protein III, respectively.

By SOE-PCR, the purified fragments G and H were linked together via their complementary ends to yield fragments GH (1.4 kb). After purification, fragment GH was digested with HindIII and EcoRI, purified and ligated into the corresponding sites of the N.BstNBI-deficient Litmus 39 vector. The resulting vector, pK10 (4.0 kb), was used to transform E. coli TG1 cells and the correctness of the DNA sequence between the HindIII and EcoRI sites was assessed by automated DNA sequencing.

Vector pK4 was constructed similarly to pK10, except that the template phagemids for PCR amplification of fragments G and H were replaced by the pK2 vector. The resulting phagemid, pK2 (4.0 kb), allows directional cloning of DNA fragments as SfiI/KpnI inserts between the regions encoding domains 2 and 3 of pIII. The correctness of the DNA sequence between the HindIII and EcoRI sites was assessed by automated DNA sequencing.

For repertoire cloning, pK10 was further engineered by cassette mutagenesis. Two 5′-phosphorylated oligonucleotides, LJ749 and LJ750 (100 pmol each, see Table 1) were annealed in a 200 μl reaction volume and directly ligated into the XhoI/NotI restriction sites of pK10. The resulting vector, pK10-AmbS (4.0 kb) contains one amber codon between the multiple cloning site and gene III. A second vector, pK10-2AmbS, carrying two amber codons at the same site was obtained by ligating two annealed oligonucleotides, LJ753 and LJ754 (see Table 1), at the same restriction sites. Both vectors were used to transform E. coli TG1 cells and the correctness of the DNA sequence between the XhoI and NotI sites was assessed by automated DNA sequencing

2. Concatenation of a Synthetic Mini-Repertoire of Human VH Fragments

A DNA fragment encompassing the randomised V_{H}-CDR2 of a synthetic repertoire of human ScFV, pIT2-1 repertoire (Tomlinson et al., unpublished) was amplified by PCR using LJ703 and LJ704 as backward and forward primers respectively. After digestion with SfiI and KpnI, the purified insert was ligated into the corresponding restriction sites of pK4. In the resulting vector, the 78-bp randomised insert is encompassed by a N.BstNBI restriction site and an AGT triplet at the 5′-end, and an ACT triplet and a N.BstNBI site at the 3′-end. After transformation of E. coli TG1 cells, approximately 10³ transformants were obtained, wherein ≧95% had the correct size insert. Plasmid DNA was then prepared from the pooled transformants.

Typically, each cycle of concatenation comprised the following steps: (1) nicking: 7.5 μg DNA was incubated with 15 U N.BstNBI (New England Biolabs 607S) in a 150 μl reaction volume at 55° C. for 2 hours; (2) elongation: after purification with the QIAquick PCR Purification kit (Qiagen 28104), 25 μl of nicked DNA is diluted with 50 μl elongation solution (15 mM Tris-HCl, pH 8.0, 15 mM MgCl₂, 1.5 mM dithiothreitol, 75 mM NaCl, 375 μM of each dNTP, 150 μg/ml bovine serum albumin) and reacted with 25 U Klenow enzyme (Boehringer 104523) at 37° C. for 1 hour; (3) ligation: after purification (optional) from 1% agarose gel with QIAquick Gel Extraction kit (QIAgen 28704), the elongated DNA was diluted to a concentration of 1-5 μg/ml in a ligation mixture containing 800 U T4 DNA ligase (New England Biolabs 202S) and further incubated at 16° C. for 3 hours prior to transformation of E. coli TG1 cells. Transformants were either pooled to prepare plasmid DNA for the next cycle of concatenation, or analysed individually by (1) PCR screening using LJ706 and LJ707 (see Table 1) as backward and forward primers, respectively, (2) by restriction pattern analysis after incubation with SpeI, and (3) by automated DNA sequencing using LJ706 for the PCR-sequencing reaction.

3. Construction of Encoded Polypeptide Repertoires

The repertoire of 1×15-mer encoded polypeptides was constructed following to a cassette mutagenesis approach using degenerated NNK triplets. Thus, a 84 bp synthetic oligonucleotide, LJ775 (2 nmole in 200 μl), encoding the bottom strand of the cassette was annealed with an extension oligonucleotide, LJ746 (4 nmole in 200 μl), at 55° C. for 30 minutes, and allowed to cool down to room temperature. The 84-bp oligonucleotide comprises the 45-bp randomised region encompassed by two antiparallel N.BstNBI sites and appropriate restriction sites for cloning. This annealed mix was diluted with 0.5 ml elongation solution (see above) and 75 μl water, and incubated with 100 U Klenow enzyme (Boehringer 104523) at room temperature for 45 minutes. After purification by phenol extraction and isopropanol precipitation (PEIP), the double-stranded DNA cassette was resuspended in 0.6 ml of 10 mM Tris, pH 8.5. Approximately 300 μl were then sequentially digested with XhoI (400 U) and NsiI (400 U), each step at 37° C. for 16 hours, followed by PEIP purification. On the other hand, 35 μg of CsCl-purified pK10-AmbS or pK10-2AmbS vector were digested with 60 U PstI at 37° C. for 2 hours, PEIP purified, digested with 240 U XhoI at 37° C. for 3 hours, and finally purified using the QIAquick PCR Purification kit (4 columns per sample). Each ligation comprised 0.75 μg insert, 10 μg vector in a 250 μl ligation mix containing 2.6 kU T4 DNA ligase. After overnight incubation at 16° C., the ligation products were submitted to restriction with 120 U PstI for counterselection of parental vector, purified as described (Kobori & Nojima, 1993), and resuspended in 25 μl water. Samples (1 μl) were electroporated (Dower et al., 1988) into 100 μl aliquots of electrocompetent E. coli TG1 cells, yielding two repertoires: one comprising 8.8×10⁷ clones (with pK10-AmbS) and one comprising 1.6×10⁸ clones (with pK10-2AmbS) on 2XTY agar plates supplemented with 5% (w/v) glucose and 100 μg/ml ampicillin (2XTYAG). The pK10-1×15-mer repertoires were scraped off the agar plates, pooled and resuspended in 2XTYAG medium at a final concentration of 40 A₆₀₀ per ml, diluted with an equal volume of sterile glycerol, and stored at −80° C. as repertoire stock. The repertoire of 1×6-mer encoded polypeptides was constructed using the same approach as for the pK10-1×15-mer repertoire, except that primer LJ775 was replaced by primer LJ748.

To construct the duplicated (or dimeric) polypeptide repertoire, a 1 ml aliquot of each of the repertoires was used to inoculate 0.4 L of 2XTYAG medium. After overnight growth at 30° C., plasmid DNA was prepared using the QIAgen Plasmid Midiprep kit (Qiagen 12143) and resuspended in 300 μl of 10 mM Tris, pH 8.5. Large-scale nicking was achieved by incubating 25 μg DNA with 50 U N.BstNBI in a 0.5 ml reaction volume at 55° C. for 3 hours. After purification with the QIAquick PCR Purification kit, the eluted DNA was diluted to 250 μl and mixed with 375 μl of elongation solution, and incubated with 40 U Klenow enzyme at 37° C. for 1.5 hour. After PEIP, 6 μg of elongated DNA was ligated in a 1.3 ml reaction volume with 10 kU T4 DNA ligase at 16° C. for 16 hours. DNA purification, electroporation, plating were then performed as described above.

To construct the tetraplicated (or tetrameric) polypeptide repertoire, the same procedure was repeated using DNA purified from the encoded duplicated repertoires, up to the purification of the ligated DNA. Thereafter, the ligated insert was amplified by PCR to allow cloning into the phage Fd-Tet-SN vector. Thus, 60 ng of purified ligated DNA was used as template for PCR (100 μl volume) with LJ793 (or LJ794 if the backbone vector is pK10-2AmbS) and LJ795 as backward and forward biotinylated primers, respectively. The DNA was purified from an agarose gel using the QIAquick Gel-Extraction kit and used as template (4 μl) for a second PCR-amplification (200 μl volume) using the same combination of primers. After PEIP, the DNA was resuspended into 50 μl of 10 mM Tris, pH 8.5, digested by XhoI (200 U at 37° C. for 16 hours) followed by SfiI (150 U at 50° C. for 7 hours). After PIEP, the DNA was resuspended into 150 μl of 10 mM Tris, pH 8.5 supplemented with 1 M NaCl, incubated with 65 μl streptavidin-coated magnetic beads (Dynal) for 30 minutes, and desalted on a Chroma Spin+TE-30 column (Clontech K1321-1). On the other hand, 50 μg of CsCl-purified Fd-Tet-SN vector were digested with 400 U XhoI at 37° C. for 3 hours followed by 400 U SfiI at 50° C. for 3 hours, and finally PEIP purified. Each of the ligation comprised 0.2 μg insert, 4 μg vector in a 200 ill ligation mix containing 2 kU T4 DNA ligase. After overnight incubation at 16° C. and counter-selection with PstI (100 U at 37° C. for 3 hours), the ligated DNA were purified as described (Kobori & Nojima, 1993), and resuspended in 25 μl water. Samples (1 μl) were electroporated (Dower et al., 1988) into 100 μl aliquots of electrocompetent E. coli TG1 cells, and plated on 2XTY agar plates supplemented with 15 μg/ml tetracycline (2XTYT). The repertoire was scraped off the agar plates, resuspended in 2XTYT medium at a final concentration of 40 A₆₀₀ per ml, diluted with an equal volume of sterile glycerol, and stored at −80° C. as polypeptide repertoire stock.

The 1×6-mer, 2×6-mer, 1×15-mer and 2×15-mer repertoires cloned into the pK10 phagemids were also subcloned in the Fd-Tet-SN phage vector. The experimental steps were identical to those described above, except that the DNA templates used for the PCR reactions were obtained from plasmid preparation of the repertoires instead of ligated DNA.

4. Construction and Selection of a Library of Duplicated E. coli Genomic Segments

The vector pW564 was constructed by replacing the NcoI-BsmI segment of pK10-AmbS with a NcoI-PstI fragment containing barnase mutant H102A (Riechmann & Winter 2000), followed by a PstI-NotI cloning region CTGCAGAGCCAGCAGACTGGCTGAGGCCTGTAACCAGTCTGCTGGATCAGCGGCCGC, then by a NotI-BsmI fragment of pIII preceded by an amber stop codon.

E. coli genomic DNA (160 μg/ml) was digested with DNase 1 (2 units/ml) for various times, the reactions stopped with EDTA, then DNA fragments of 100-270 bp from the 30 and 40 minute timepoints isolated by agarose gel electrophoresis and purified with QIAquick columns (QIAGEN). 150 ng of this size-selected DNA was used as a template in a 200 μl random amplification (250 μM dNTPs, 2 μM primer BXN6, 100 units/ml Taq, in the manufacturer's buffer), cycling program 94° 5′; 30×[94° 1′; 4° 1′; 25° 5′; 30° 5′; 35° 5′; 72° 0.5′]; 70° 5′; 4° hold. The reaction was purified on QIAquick columns and 200 μl was used as template in a 2000 μl PCR with constant sequence primer BXLONG, cycling program 94° 2′; 35×[95° 1′; 65° 1′; 72° 20″]; 72° 5′; 4° hold. The PCR was purified by PEIP and digested with BstXI, electrophoresed, and fragments of 160-250 bp were purified. The vector pW564 was digested with BstXI and StuI, purified by PEIP and spin-column (Chromaspin-1000, Millipore). Vector and insert were ligated at a molar ratio of 4 and vector concentration of 12 μg/ml, then purified by PEIP and ultrafiltration (Microcon, Millipore). ˜40 μg of ligation product was electroporated into E. coli strain WX109 (a male derivative of strain MC1061), resulting in 1.4×10⁹ transformants. This was called library#4.

Library#4 was subjected to a selection for inserts in-frame with pIII. 1.1×10¹⁰ bacteria from library#4 were expanded and infected with helper phage KM13supF (a derivative of KM13 (Kristensen & Winter 1998) carrying a supF tRNA) then grown to saturation in 2×TY+Amp+Kan; phage were purified from the supernatant by PEG precipitation. 1.4×10¹¹ phage were digested with trypsin (1 mg/ml in PBS) for 5 minutes, then infected into E. coli strain XL1-Blue. This was called library#6. DNA sequencing showed that 16/18 clones with inserts were in-frame with pIII and without stop codons (compared to 2/19 clones from library#4). To remove clones without inserts, library#6 was re-cloned. The inserts were amplified with the primers barnBa1/BxNotFo, digested with PstI+NotI, electrophoresed, and fragments of 150-280 bp were purified. These insert fragments were ligated to pW564 (purified by caesium chloride ultracentrifugation) then digested with PstI+NotI. 50 μg of ligation product was electroporated into WX109 bacteria, and resulted in 1.3×10¹⁰ transformants. This was called library#7. Nearly all clones had inserts (31/32 by PCR screen, compared to 19/32 in library#6).

Library#7 was submitted to one cycle of concatenation. 300 μg/ml of library#7 plasmid DNA was treated with 600 units/ml N.BstNBI in the manufacturer's buffer at 55° C. for 90 minutes, and purified by PEIP. This nicked DNA at 100 μg/ml was incubated with 40 units/ml Klenow fragment and 300 μM dNTPs in the manufacturer's buffer at 37° C. for 75 minutes then at 50° C. for 20 minutes, purified by PEIP, electrophoresed and the desired extension product (the major band of 4.5 kb) purified. The purified product, at 3.75 μg/ml, was incubated with 2 units/μl T4 DNA ligase in the manufacturer's buffer at 16° C. overnight, and purified by PEIP and ultrafiltration. ˜7.5 μg of ligation product was electroporated into WX109, and resulted in 6.0×10⁹ transformants. This was called library#8.

Library#8 was subjected to proteolytic selection. 8×10¹⁰ bacteria from library#8 were grown and infected with KM13supF; phage were purified from the supernatant by two PEG precipitations and resuspended in PBS+15% glycerol. Streptavidin-coated wells (Streptawell HiBind, Roche) were pre-coated with 200 μl biotinylated barstar (1 μg/ml in PBS+0.1% BSA) at room temperature for 1 hour, washed twice with PBS, then blocked with PBS+6% skimmed milk (PBS6M). 200 μl phage was mixed with 400 μl 1.5×TBSC+7.5% glycerol (TBSC=25 mM Tris-HCl pH 7.4, 137 mM NaCl, 1 mM CaCl₂) and equilibrated to 10° C. Trypsin and thermolysin were added to final concentrations of 200 nM and 384 nM, respectively, and incubated at 10° C. for 10 minutes. Protease inhibitors Pefabloc (Boehringer-Mannheim) and 1,10-phenanthroline were each added to 100 μM; after 2 more minutes, 400 μl PBS6M was added. 200 μl aliquots of the proteolyzed phage were applied to barstar-coated wells and incubated at room temperature for 1 hour. Wells were washed four times with PBS, once with PBS+50 mM DTT (incubated for 5′), four times with PBS+1 mM DTT, and once with PBS. Bound phage were eluted with 200 μl of 100 mM glycine pH 2.1 for 5 minutes; the eluate was neutralised by mixing with 200 μl 1M Tris pH 8. The eluted phage were infected into WX109. From an input of 2.2×10¹² phage, 1.0×10⁷ phage were recovered. After bacterial growth, plasmid DNA was prepared and inserts were PCR-amplified, gel-purified, and re-cloned into pW564 (to remove clones without inserts). Phage were prepared from this and PEG purified. A second round of proteolytic selection was performed similar to round one; from an input of 1.1×10¹¹ phage, 3.2×10⁷ phage were recovered.

Phage-containing supernatants from individual clones arising after round 2 were screened for protease resistance. Streptawells were pre-coated with biotinylated barstar and blocked as above. 75 μl PBS6M and 25 μl phage supernatant were applied to each well and incubated for 1 hour. Wells were washed with PBS and TBSC. 100 μl TBSC or protease solution (200 nM trypsin, 384 nM thermolysin, in TBSC) was applied and incubated for 10 minutes at room temperature. Wells were washed three times with PBS, once with PBS+56 mM DTT (incubated for 5 minutes), three times with PBS+1 mM DTT, and once with PBS. 100 μl HRP-anti-phage conjugate (1:5000 dilution in PBSM, Pharmacia) was applied and incubated for 1 hour. Wells were washed with PBST and PBS. 100 μl tetramethylbenzidine substrate was applied, and stopped with 50 μl 1M sulphuric acid; absorbances were measured at 450 nm with subtraction of 650 nm background. Protease resistance was calculated as the ELISA signal with protease treatment divided by the signal without protease treatment.

5. Concatenation of Linear DNA Fragments Anchored to a Surface

Linear DNAs were prepared by PCR amplification of bacteria for clones 6.1 and 6.12 using versions of primers barnBa1 and g3seq6 having biotin at the 5′ end, followed by QIAquick column purification. Streptavidin-coated magnetic particles (Dynabeads M-280 Streptavidin, Dynal) were washed with 2×BWB (10 mM Tris pH 7.4, 1 mM EDTA, 2M NaCl). 1.5 μl PCR product (˜10 fmoles) was applied to 100 μl beads in 1×BWB, and incubated with shaking for 15′ at room temperature. After washing twice with 2×BWB, half was saved as untreated beads. The other half was washed with 1×N.BstNBI buffer and resuspended in 20 μl nicking mix (1×N.BstNBI buffer, 100 μg/ml BSA, 0.5 units/I N.BstNBI) and incubated at 55° C. for 30 minutes, vortexing every 10 minutes. After washing twice with 2×BWB and once with 1×EcoPol buffer, the beads were resuspended in 20 μl extension mix (1×EcoPol buffer, 100 μg/ml BSA, 250 μM dNTPs, 0.5 units/μl Klenow) and incubated at 37° C. for 30 minutes, vortexing every 10 minutes. After washing twice with 2×BWB and once with 1× Ligase buffer, the beads were resuspended in ligation mix (1× Ligase buffer, 100 μg/ml BSA, 40 units/μl T4 DNA ligase) and incubated with shaking at room temp for 2.5 hours. Beads were washed once with 2×BWB and resuspended in TE; untreated beads were also resuspended in TE. 1.5 μl beads were used as template for a 30 μl PCR with primers barnBa1/BxNotFo. PCRs were purified on QIAquick columns and examined by agarose gel electrophoresis.

6. Preparation of EC-EpoR Products

For biopanning, a fusion protein comprising the extracellular domain of human erythropoietin receptor (EC-EpoR, amino acids 1 to 225) was fused to the Fc portion of human IgG1, and expressed as a 110 kD (2×55 kD) recombinant protein in Pichia pastoris. The gene encoding EC-EpoR-Fc was constructed by SOE-PCR:

Fragment A was amplified from pSVsport-EpoR/IFNαR2 (gift from Dr. J. Tavernier, Ghent, Belgium) by PCR using LJ764 and LJ767 (see Table 1) as backward and forward primers, respectively. In addition to a 5′-end extension containing a XhoI restriction site, the resulting DNA fragment contains a silent G->A substitution at triplet encoding Glu60, which destroys an internal XhoI restriction site in the EpoR gene.

Fragment B was amplified from pSVsport-EpoR/IFNαR2 by PCR using LJ766 and LJ761 (see Table 1) as backward and forward primers, respectively. In addition to a 3′-end extension encoding a Gly-Thr-Gly-Ser-Gly-Ser-Ala linker, the resulting DNA contains fragment G->A substitution at triplet encoding Glu60 in the EpoR gene.

Fragment C was amplified from pMac-Id/Fc (gift from Dr. J. Tavernier) by PCR using LJ762 and LJ765 (see Table 1) as backward and forward primers, respectively. In addition to a 3′-end extension containing a XbaI restriction site, the resulting DNA fragment contains the 5′-end extension encoding a Gly-Thr-Gly-Ser-Gly-Ser-Ala linker.

By SOE-PCR, the purified fragments A, B and C were linked together via their complementary ends to yield fragment ABC (1.4 kb). After purification and digested with XhoI and XbaI, the purified fragment was ligated into the corresponding sites of pPICZαA (Invitrogen V195-20) which directs expression of heterologous genes for secretion. After transformation of E. coli TG1, cells, individual transformants on Zeocin-selective agar plates were detected by PCR screening using LJ780 and LJ779 as backward and forward primers, respectively, and positive clones were further analysed by automated DNA sequencing. Next, the whole procedure aiming at preparing DNA for transformation of Pichia X-33 cells, transformation, screening and expression was done strictly according to the manufacturer's instructions in the EasySelect Pichia Expression kit (Invitrogen K1740-01).

The chimaeric EC-Epor-Fc protein was purified from a 250 ml culture supernatant after 24 hours of induction with 1% (v/v) methanol at 30° C. Briefly, after pH adjustment to 7.0, the sterile filtered supernatant was loaded onto a HiTrap Protein A column (Amersham Pharmacia Biotech 17-0402-01) at a flow rate of 1 ml per minute. Column washing and elution of bound protein was done according to the manufactuer's instruction, yielding about 4.7 mg protein. Desalting and further purification from truncated products was achieved by size-exclusion chromatography on a Superdex 75 HR 10/30 column (Amersham Pharmacia Biotech 17-1047-01) in PBS at a flow rate of 0.5 ml per minute, yielding 2.6 mg protein. Non-reducing SDS-page analysis on 10% Bis-Tris gel (Novex NP0302) revealed a predominant 110 kD product corresponding to an homodimeric EC-EpoR-Fc linked via two disulphide bonds at the IgG1 hinge, whereas a weak band at 55 kD suggested that not all EC-EpoR-Fc form a covalent product.

For the biopanning experiments, EC-EpoR-Fc was biotinylated according to the Biotin Protein Labelling kit (Roche Molecular Biochemicals 1418165) but using the biotin disulphide N-hydroxysuccinimide ester (Sigma B4531) as reagent.

For ELISA analysis, a monomeric EC-EpoR-Fc construct was produced by replacing the two Cys residues at the IgG1 hinge with Ser residues. Briefly, the Ec-EpoR gene was PCR amplified from pPICZαA-EC-EpoR-Fc with primers LJ764 (backward) and LJ871 (forward, adapted for mutagenesis of the Cys residues in the IgG1 hinge), and the IgG1 gene was PCR amplified from pPICZαA-EC-EpoR-Fc with primers LJ870 (backward, adapted for mutagenesis of the Cys residues in the IgG1 hinge) and LJ765 (forward). After purification, the DNA fragments were linked by SOE-PCR using primers LJ764 and LJ765. Next, the DNA fragment was digested with XbaI and XhoI, ligated into the corresponding sites of pPICZαA (Invitrogen V195-20). After transformation of E. coli TG1 cells, individual transformants on Zeocin-selective agar plates were detected by PCR screening using LJ780 and LJ779 as backward and forward primers, respectively, and positive clones were further analysed by automated DNA sequencing. Protein expression and purification was subsequently performed as described above. In addition, a truncated construct was produced by cloning the DNA fragment encoding the IgG1 hinge (with the Cys residues) and the Fc portion. This DNA fragment was produced by PCR amplification from pPICZαA-EC-EpoR-Fc with primers LJ869 (backward) and LJ765 (forward), digested with XbaI and XhoI, ligated into pPICZαA before transformation of E. coli TG1 cells. Clone characterisation, protein expression and purification were performed as described above.

7. Selection of Encoded Polypeptide Repertoires on EC-EpoR-Fc

Phage were produced from the 1×15-mer, 2×15-mer and 4×15-mer encoded polypeptide repertoires by inoculating 0.5 L of 2XTYT medium with 0.5 ml repertoire stock, incubating at 37° C. for 20 hours. Phage particles were PEG-precipitated from the cleared supernatants, resuspended in PBS supplemented with 5% glycerol, and stored at −20° C. For each biopanning, 10¹² TU were diluted in 0.3 ml of PBS supplemented with 2% skimmed milk (PBSM) and biotinylated EC-EPOR-Fc (0.25 μM final concentration) and incubated at 4° C. for 3 hours. Next, the phage solution was reacted with 100 μl of streptavidin-coated beads (Dynal) preblocked with PBSM at 4° C. for 1 hour. After ten washes with 0.5 ml of cold PBS containing 0.1% Tween-20, the beads were resuspended in 300 μl of 50 mM DTT in PBS and incubated for 10 minutes with agitation. The supernatant containing the eluted phage was diluted to 1 ml with 2XTY, and used to infect E. coli TG1 cells as described (Marks et al., 1991) for amplification before the second selection round. The selection procedure for the second and third rounds of selection were similar to that of the first selection round, except that (1) the input of phage particles was reduced to 10¹¹ TU, and (2) the final concentration of biotinylated EC-EpoR-Fc was reduced to 25 nM but only in the third selection round. Individual clones recovered after the second and the third rounds of selection were then analysed by phage-ELISA.

8. Selection of Encoded Polypeptide Repertoires on Ferritin

Phage were produced from the 1×6-mer, 2×6-mer, 4×6-mer, 1×15-mer, 2×15-mer and 4×15-mer encoded polypeptide repertoires by inoculating 0.5 L of 2XTYT medium with 0.5 ml repertoire stock, incubating at 37° C. for 20 hours. Phage particles were PEG-precipitated from the cleared supernatants, resuspended in PBS supplemented with 5% glycerol, and stored at −20° C. For each biopanning, 10¹² TU were diluted in 0.3 ml of PBS supplemented with 2% skimmed milk (PBSM) and ferritin-biotinamido-caproyl (0.25 μM final concentration, Sigma cat. F3652) and incubated at 4° C. for 3 hours. Next, the phage solution was reacted with 100 μl of streptavidin-coated beads (Dynal) preblocked with PBSM at 4° C. for 1 hour. After ten washes with 0.5 ml of cold PBS containing 0.1% Tween-20, the beads were resuspended in 800 μl of 0.1 M glycine-HCl, pH 2.1 and incubated for 10 minutes with agitation. The supernatant containing the eluted phage was recovered, neutralised with 200 μl of 1 M Tris, pH 8.0, and used to infect E. coli TG1 cells as described (Marks et al., 1991) for amplification before the second selection round. The selection procedure for the second and third rounds of selection were similar to that of the first selection round, except that (1) the input of phage particles was reduced to 10¹¹ TU, and (2) the final concentration of biotinylated ferritin was reduced to 25 nM but only in the third selection round. Individual clones recovered after the second and the third rounds of selection were then analysed by phage-ELISA.

9. ELISA

For phage-ELISA, EC-EpoR-Fc or ferritin (from horse spleen, Sigma cat. F-4503) was coated overnight in high-binding microtitre plates (Costar) at 4° C. (1 μg protein in 100 μl PBS per well). After blocking with PBSM at 4° C. for 3 hours, the wells were incubated for two hours with either 50 μl crude culture supernatant (supplemented with 50 μl PBSM) or dilution series of purified phage particles in PBSM (starting from 10¹⁰ TU per well). After washing with PBST buffer, bound phage were detected by incubation with horseradish peroxidase anti-phage conjugate (1/5000 dilution, Amersham Pharmacia Biotech), and o-phenylene diamine dihydrochloride (OPD) as substrate. After 10-15 minutes, the reactions were stopped with 50 μl of 4 N H₂SO₄ and absorbances were measured at 492 nm.

For competition ELISA with synthetic trMP peptide (see below), serial dilutions of MP peptide (starting from 1 mM) were mixed with 2.5×10⁸ phage in 2% MPBS, then added to pre-blocked EC-EpoR-Fc-coated wells for 2 hours. Bound phage were detected as described above. Alternatively, serial dilutions of MP peptide (starting from 1 mM) were incubated with EC-EpoR-Fc (at 10 μg/ml) in 2% MPBS for 1 hour at 37° C., then added to ELISA wells coated with MBP-fusions (at 10 μg/ml in PBS overnight at 4° C.). Bound EC-EpoR-Fc was detected with protein A-horseradish peroxidase conjugate (1/4000 dilution, Sigma), and o-phenylene diamine dihydrochloride (OPD) as substrate. After 10-15 minutes, the reactions were stopped with 50 μl of 4 N H₂SO₄ and absorbances were measured at 492 nm.

For competition ELISA with ferritin-binding peptide, serial dilutions of MBP-fusions (see below) were incubated with 10 nM biotinylated ferritin in 2% MPBS overnight at 4° C., then added to pre-blocked MBP-(HKPRKE)₈-coated wells for 30 min. Bound biotinylated ferritin was detected by incubation with streptavidin horseradish peroxidase conjugate (Sigma) and OPD.

10. Alanine-Scanning Mutagenesis on MP Sequence

Alanine-mutants were first constructed in the monomeric sequence of the selected 2×15-mer polypeptide on EC-EpoR-Fc. For each alanine-mutant, two oligonucleotides (40 mole each) were annealed at 55° C. for 30 minutes, and allowed to cool down to room temperature (LJ798 and LJ799 for wild-type, LJ803 and LJ806 for P1A, LJ804 and LJ806 for L2A, LJXXX and LJXXX for C4A, LJ805 and LJ806 for H5A, LJ807 and LJ808 for T8A, LJ807 and LJ809 for L9A, LJ807 and LJ810 for E10A, LJ807 and LJ811 for Q11A, LJ807 and LJ812 for T12A, LJ807 and LJ813 for Y13A, LJ807 and LJ814 for L15A). This annealed mixes were diluted with 60 μl elongation solution (see above) and incubated with 20 U Klenow enzyme at 37° C. for 1 hour. In the resulting double-stranded DNA cassettes, the sequence encoding the monomeric peptide motif is encompassed by two antiparallel N.BstNBI sites (as in the original repertoire) and two restriction sites: NsiI and XhoI at the 5′ and 3′-end, respectively. After purification by phenol extraction and isopropanol precipitation (PEIP), the double-stranded DNA cassette were overnight digested with 40 U XhoI and 40 U NsiI at 37° C. for 16 hours, followed by PEIP purification, and ligation into pK10-2AmbS. After transformation of E. coli TG1 cells and PCR screening with LJ008 and PST216, positive clones were submitted to automated DNA sequencing. To obtain single alanine mutant in the 2×15-mer peptide sequence, a procedure similar to that described in Concatenation of a synthetic mini-repertoire of human VH fragments was used, with two exceptions: (1) after the nicking step, each aliquot of mutant vector was mixed with an equimolar amount of wild-type vector, incubated at 98° C. for 4 minutes, and then cooled on ice. This step aims at denaturing the nicked vectors and reanneal them such that statistically the top and bottom strands of both vectors will be exchanged before the elongation step. As a result, there will be a 25% probability that a given alanine mutation will be found either within the first half or within the second half of the 2×15-mer polypeptides after elongation and ligation; (2) after ligation, the inserts were directly amplified by PCR with primers LJ008 and LJ795 to allow cloning into the phage Fd-Tet-SN vector. After purification from an agarose gel using the QIAquick Gel-Extraction kit and digestion with SfiI and XhoI, the inserts were ligated into the corresponding sites of Fd-Tet-SN. Following transformation of E. coli TG1 cells, individual clones were analysed by PCR screening with primers LJ006 and PST216 for correct size inserts, and several positive clones from each transformation were submitted to automated DNA sequencing.

11. Production of Synthetic Truncated MP Peptide

A 26-amino acid cyclic peptide (NH₂-ACHGATLEQTYALPL ACHGATLEQTY-CO₂H) was obtained from Peptide Products (UK). Mass-spectrometric analysis by laser desorption and HPLC in 80% acetonitrile/0.1% TFA revealed a >95% pure product of 2761.52 D (predicted MW: 2761.18 D) which was tested Ellman negative.

12. Production of Recombinant Peptide Fusions

Selected peptides on EC-EpoR-Fc and on ferritin, mutants thereof, concatenated versions thereof and the EMP1 peptide (Wrighton et al., 1996) were obtained as maltose-binding protein fusions (MBP-fusions). Briefly, DNA fragments encoding the peptides were cloned as DNA cassette into pMAL-p2x (for cyclic peptides) or pMAL-c2x (for linear peptides) (New England Biolabs) together with a C-terminal hexa-histidine tag. Transformation of E. coli TB1 cells, cell growth and protein expression were performed according to the manufacturer's instructions. MBP-fusions were purified (>90% homogeneity) from periplasmic preparations (for cyclic peptides) or sonicated extracts (for linear peptides) on HiTrap chelating columns (Pharmacia). The purified peptide fusions were dialysed overnight at 4° C. in PBS, before subsequent analysis.

13. Determination of Apparent Dissociation Constants

Apparent dissociation constants were calculated using the BIAevaluation program and the 1:1 Langmuir dissociation model from sensorgrams (BIAcore) collected upon immobilisation of 200-400 resonance units (RU) of biotinylated ligands (sEPOR-Fc or MBP-fusion) on SA-sensor chips followed by injection of various analytes (sEPOR-Fc, MBP-fusion, 0.1 to 2 μM concentration range) diluted in HBS-EP buffer (BIAcore), at a flow rate of 80 μl/min at 25° C.

TABLE 1

Sequence Listing of all oligonucleotides used in this invention (all
written from 5′- to 3′-end, P-: phosphate group, B-: biotin group)

| | |
|---|---|
| PST216: | ATG GGG TTT TGC TAA ACA ACT TTC |
| LJ005: | ATG GTT GTT GTC ATT GTC GGC GCA |
| LJ008: | CAG GAA ACA GCT ATG AC |
| LJ667: | GAC ACG CGT CTC AGC CAG GCA ACT ATG GAT GAA CGA |
| LJ668: | GTC CAC GTT CTT TAA TAG TGG ACA CTT GTT CCA AAC TGG AAC |
| LJ669: | GTC CAC TAT TAA AGA ACG TGG ACA CCA ACG TCA AAG GGC G |
| LJ670: | GAC ACG CGT CTC TTC AGT CCA ACC CGG TAA GAC ACG AC |
| LJ671: | GAC ACG CGT CTC ACT GAA GAC GAT AGT TAC CGG ATA AGG CG |
| LJ674: | GAC ACG CGT CTC CTG GCT CCC CGT CGT GTA GAT AAC TAC G |
| LJ675: | CAG TGA ATT CCC ACC TGC AGC CAA GCT TGG CGT AAT CAT GGT CAT AGC |
| | TG |
| LJ676: | CCA AGC TTG GCT GCA GGT GGG AAT TCA CTG GCC GTC GTT TTA CAA CGT |
| | CGT G |
| LJ678: | ATT AAG AGG CTG AGA TTC CTC AAG AGA AGG |
| LJ679: | CCT TCT CTT GAG GAA TCT CAG CCT CTT AAT |
| LJ680: | CGG CGA ATT CTT ATT AAG ATT CCT TAT TAC GCA GTA TGT TAG CAA ACG |
| | TGC |
| LJ703: | CGC ACT GGC GGC CCA GCC GGC CCT GAG TCA GCT AGT GGG AAG GGG CTG |
| | GAG TGG GTC TCA |
| LJ704: | GCC GCC GGT ACC GAG TCC AGC AGT CCG GCC CTT CAC GGC GTC TGC GTA |
| LJ706: | GGT AAA TTC AGA GAC TGC GCT TTC |
| LJ707: | ATT TTC GGT CAT AGC CCC CTT ATT AGC |
| LJ746: | TGC TGC ATG CAT GAG TCC GGT |
| LJ748: | GCT CCG CTC GAG TCC AGA MNN MNN MNN MNN MNN MNN ACC GGA CTC ATG |
| | CAT CGA GCA |
| LJ749: | P-TCG AGC AGA TCT AGT CTG C |
| LJ750: | P-GGC CGC AGA CTA GAT CTG C |
| LJ753: | P-TCG AGC AGT AGT AGT CTG C |
| LJ754: | P-GGC CGC AGA CTA CTA CTG C |
| LJ761: | CGG CCG AAC CAC TTC CGG TAC CGT CCA GGT CGC TAG GCG TCA GCA G |
| LJ762: | CGG TAC CGG AAG TGG TTC GGC CGA GCC CAA ATC TTC TGA CAA AAC TCA C |
| LJ764: | GAC GCA CGC CTC GAG AAA AGA GCG CCC CCG CCT AAC CTC CCG GAC |
| LJ765: | CTA GTC TAG AGC TTT ACC CGG AGA CAG GGA GAG GCT C |
| LJ766: | CTC CTA CCA GCT CGA AGA TGA GCC ATG G |
| LJ767: | CCA TGG CTC ATC TTC GAG CTG GTA GGA G |
| LJ775: | GCT CCG CTC GAG TCC AGA MNN MNN MNN MNN MNN MNN MNN MNN MNN MNN |
| | MNN MNN MNN MNN MNN ACC GGA CTC ATG CAT CGA GCA |
| LJ779: | GCA AAT GGC ATT CTG ACA TCC |
| LJ780: | TAC TAT TGC CAG CAT TGC TGC |
| LJ793: | B-CTC GCA CTC GCG GCC CAG CCG GCC ATG GCC CAG |
| LJ794: | B-GGC CGC AGA CTA CTA CTG CTC GAG TCC AGA |
| LJ795: | B-GGC CGC AGA CTA GAT CTG CTC GAG TCC AGA |
| LJ798: | B-TGC TGC ATG CAT GAG TCC GGT CCT CTG GCT TGT CAT GGT GCG ACG TTG |
| | GAG |
| LJ799: | B-GCT CCG CTC GAG TCC AGA CAG AGC ATA TGT CTG CTC CAA CGT CGC ACC |
| | ATG |
| LJ801: | TGC TCT AGA GAG GTG GAG CTG CAT GAG TCC GGT |
| LJ802: | CAC AAG CTT TTA CGC CCG TTT GAT CTC GAG TCC AGA |
| LJ803: | TGC ATG CAT GAG TCC GGT GCT CTG GCT TGT CAT GGT GCG ACG TTG GAG |
| | GAG |
| LJ804: | TGC ATG CAT GAG TCC GGT CCT GCG GCT TGT CAT GGT GCG ACG TTG GAG |
| | CAG |
| LJ805: | TGC ATG CAT GAG TCC GGT CCT CTG GCT TGT GCT GGT GCG ACG TTG GAG |
| | CAG |
| LJ806: | GCG CTC GAG TCC AGA CAG AGC ATA TGT CTG CTC CAA CGT CGC ACC |
| LJ807: | TGC ATG CAT GAG TCC GGT CCT CTG GCT TGT CAT GGT GCG |
| LJ808: | GCG CTC GAG TCC AGA CAG AGC ATA TGT CTG CTC CAA CGC CGC ACC ATG |
| | ACA AGC CAG |
| LJ809: | GCG CTC GAG TCC AGA CAG AGC ATA TGT CTG CTC CGC CGT CGC ACC ATG |
| | ACA AGC CAG |
| LJ810: | GCG CTC GAG TCC AGA CAG AGC ATA TGT CTG CGC CAA CGT CGC ACC ATG |
| | ACA AGC CAG |
| LJ811: | GCG CTC GAG TCC AGA CAG AGC ATA TGT CGC CTC CAA CGT CGC ACC ATG |
| | ACA AGC CAG |
| LJ812: | GCG CTC GAG TCC AGA CAG AGC ATA CGC CTG CTC CAA CGT CGC ACC ATG |
| | ACA AGC CAG |
| LJ813: | GCG CTC GAG TCC AGA CAG AGC CGC TGT CTG CTC CAA CGT CGC ACC ATG |
| | ACA AGC CAG |
| LJ814: | GCG CTC GAG TCC AGA CGC AGC ATA TGT CTG CTC CAA CGT CGC ACC ATG |
| | ACA AGC CAG |
| BXN6: | CTT CCA GCA GAC TGG GAG TCA NNN NNN |
| BXLONG: | GTG GAT ACG AAC TTC CAG CAG ACT GGG AGT CA |
| barnBa1: | GCT TAT CAG ACC TTT ACA |
| g3seq6: | CCC TCA TAG TTA GCG TAA CGA |
| BxNotFo: | TCT ATG CGG CCG CTG ATC C |

### REFERENCES

Arlidn, A. P. and Youvan, D. C. (1992) Proc Natl Acad Sci USA. 89:7811-5.

Berman, A. L., Kolker, E. and Trifonov, E. N. (1994) Proc. Natl. Acad. Sci. USA. 91: 4044-7.

Blundell, T. L. and Srinivasan, N. (1996) Proc. Natl. Acad Sci. USA. 93:14243-8.

Bogarad, L. D. and Deem, M. W. (1999) Proc. Natl. Acad. Sci. USA. 96:2591-5.

Brown, S. (1997) Nat. Biotechnol. 15:269-72.

Cherry, J. R., Lamsa, M. H., Schneider, P., Vind, J., Svendsen, A., Jones, A. and Pedersen, A. H. (1999) Nat. Biotechnol. 4: 379-84.

Cohen, B. and Carmichael, G. G. (1986) DNA. 5:339-43.

Coles, M., Diercks, T., Liermann, J., Groger, A., Rockel, B., Baumeister, W., Koretke, K. K., Lupas, A., Peters, J., Kessler, H. (1999) Curr. Biol. 9:1158-68.

Daugherty, P. S., Chen, G., Iverson, B. L. and Georgiou, G. (2000) Proc. Natl. Acad Sci. USA., 97:2029-34.

Davidson, A. R., Lumb, K. J. and Sauer, R. T. (1995) Nat. Struct. Biol. 2:856-63.

Douglas, T. & Ripoll, D. R. (1998) Protein Sci. 7:1083-91.

Farber, G. K. and Petsko, G. A. (1990) Trends Biochem. Sci. 15:228-34.

Fire, A. and Xu, S. Q. (1995) Proc. Natl. Acad. Sci. USA. 92:4641-5.

Goraj, K., Renard, A. and Martial, J. A. (1990) Protein Eng. 3:259-66.

Gram, H., Marconi, L. A., Barbas, C. F. 3d, Collet, T. A., Lerner, R. A. and Kang A. S. (2000) Proc. Natl. Acad. Sci. USA. 89:3576-80.

Hardies, S. C., Patient, R. K., Klein, R. D., Ho, F., Reznikoff, W. S. and Wells, R. D. (1979) J. Biol. Chem. 254:5527-5534.

Hartley, J. L. and Gregori, T. J. (1981) Gene. 13:347-53.

Hawkins, R. E., Russell, S. J. and Winter, G. (1992) J. Mol. Biol., 226:889-96.

Hecht, M. H., Richardson, J. S., Richardson, D. C. and Ogden, R. C. (1990) Science. 249:884-91.

Heringa, J. and Taylor, W. R. (1997) Curr. Opin. Struct. Biol. 7:416-21.

Hofer, B. (1987) J. Biochem. 167:307-13.

Houbrechts, A., Moreau, B., Abagyan, R., Mainfroid, V., Preaux, G., Lamproye, A., Poncin, A., Goormaghtigh, E., Ruysschaert, J. M., Martial, J. A. and Goraj, K. (1995) Protein Eng. 8:249-59.

Jiang, S. W., Trujillo, M. A. and Eberhardt, N. L. (1996) Nucl. Acids. Res., 24:3278-9.

Kim, S. C. and Szybalski, W. (1988) Gene. 71: 1-8.

Kovacs, B. J., Gregory, S. P. and Butterworth, P. H. W. (1984) Gene. 29:63-8.

Kristensen, P., Winter, G. (1998) Folding & Design. 3:321-8.

Lang, D., Thoma, R., Henn-Sax, M., Sterner, R. and Wilmanns, M. (2000) Science. 289, 1546-50.

Lapatto, R., Blundell, T., Hemmings, A., Overington, J., Wilderspin, A., Wood, S., Merson, J. R., Whittle, P. J., Danley, D. E. and Geoghegan, K. F. (1989) Nature. 342:299-302.

Livnah, O., Stura, E. A., Johnson, D. L., Middleton, S. A., Mulcahy, L. S., Wrighton, N. C., Dower, W. J., Jolliffe, L. K. and Wilson, I. A. (1996) Science. 273:464-71.

Lonberg, N. and Gilbert, W. (1985) Cell. 40:81-90.

Marcotte, E. M., Pellegrini, M., Yeates, T. O., Eisenberg, D. (1998) J. Mol. Biol. 293:151-60.

McConnell, S. J., Dinh, T., Le, M.-H., Brown, S. J., Becherer, K., Blumeyer, K., Kautzer, C., Axelrod, F. and Spinella, D. G. (1998) Biol. Chem. 379:1279-86.

Nalcamaye, K. and Eckstein, F. (1986) Nucl. Acids Res. 14:9679-98.

Osterlund, M., Luthman, H., Nilsson, S. V. and Magnusson, G. (1982) Gene. 20:121-5.

Parker, R. C., Watson, R. M. and Vinograd, J. (1977) Proc. Natl. Acad. Sci. USA. 75:2170-4.

Raclcovsky, S. (1998) Proc. Natl. Acad. Sci. USA. 95:8580-4.

Riechmann, L., Winter, G. (2000) Proc. Natl. Acad. Sci. USA. 97:10068-73.

Regan, L. and DeGrado, W. F. (1988) Science. 241:976-8.

Sadler, J. R., Betz, J. L. and Tecklenburg, M. (1978) Gene. 3:211-32.

Sadler, J. R., Tecklenberg, M. and Betz, J. L. (1980) Gene. 8:279-300.

Savageau, M. A. (1986) Proc. Natl. Acad Sci. USA. 83:1198-202.

Schafmeister, C. E., LaPorte, S. L., Miercke, L. J. W. and Stroud, R. M. (1997) Nat. Struct. Biol. 4:1039-46.

Shiba, K., Takahashi, Y. and Noda, T. (1997) Proc. Natl. Acad. Sci. USA, 94:3805-10.

Skutelsky, E. and Bayer, E. A. (1987) J. Histochem. Cytochem. 35: 1063-8.

Song J. K. and Rhee J. S. (2000) Appl. Environ. Microbiol. 66:8904.

Stemmer, W. P. C. (1994) Nature. 370: 389-91.

Taylor, J. W., Schmidt, W., Cosstick, R., Okruszek, A. and Eckstein, F. (1985) Nucl. Acids Res. 13:8749-64.

Tian, S. S., Lamb, P., King, A. G., Miller, S. G., Kessler, L., Luengo, J. I., Averill, L., Johnson, R. K., Gleason, J. G., Pelus, L. M., Dillon, S. B. and Rosen, J. (1998) Science. 281:257-9.

Taylor, W. H. and Hagerman, P. J. (1987) Gene. 53:139-144.

Tawfik, D. S. and Griffiths, A. D. (1998) Nat. Biotechnol. 16:652-6.

Walker, G. T., Little, M. C., Nadeau, J. G. and Shank, D. D. (1992) Proc. Natl. Acad. Sci. USA. 89:392-6.

Wang, B. S. and Pabo, C. O. (1999) Proc. Natl. Acad. Sci. USA. 96:9568-73.

Wende, W., Stahl, F. and Pingoud, A. (1996) Biol. Chem., 377:625-32.

White, M. J., Fristensky, B. W. and Thompson, W. F. (1991) Anal. Biochem., 1991:184-90.

Wolynes, P. G. (1996) Proc. Natl. Acad. Sci. USA. 93:14249-55.

Wrighton, N. C., Farrell, F. X., Chang, R., Kashyap, A. K., Barbone, F. P., Mulcahy, L. S., Johnson, D. L., Barrett, R. W., Jolliffe, L. K. and Dower, W. J. (1996) Science. 273:458-64.

Xu, D. and Nussinov, R. (1997) Curr. Biol. 3:11-7.

You, L. and Arnold, F. H. (1996) Protein Eng. 9:77-83.

Zhao, H., Giver, L., Shao, Z., Affholter, J. A. and Arnold, F. H. (1998) Nat. Biotechnol. 16:258-61.

Zwick, M. B., Shen, J. and Scott, J. K. (2000) J. Mol. Biol. 300:307-20.

1. A homogenous repertoire of concatenated nucleic acid sequences.

2. A repertoire of concatenated nucleic acid sequences wherein not more than two identical copies of each target nucleic acid sequence are linked together in head-to-tail orientation on the same molecule of DNA.

3. A repertoire of concatenated polypeptides encoded by the concatenated nucleic acid sequences according to claim 1 or claim 2.

4. A method for creating a concatenated repertoire of target nucleic acid sequences, comprising the step of linking not more than two identical copies of each target nucleic acid sequence in head-to-tail orientation on the same molecule of DNA.

5. A method for creating a concatenated repertoire according to claim 4, comprising using each of the two complementary strands of DNA of a target nucleic acid sequence is used as template for synthesis of a complementary strand of nucleic acid so as to generate not more than two copies of each of the target nucleic acid sequences, and ligating said copies together in a head-to-tail orientation on the same molecule of DNA.

6. An in vitro method for constructing a concatenated repertoire according to claim 4, comprising the steps of:
a) introducing two single-strand nicks into a starting repertoire of double-stranded target nucleic acid molecules and converting both strands of the starting repertoire of target nucleic acid sequences into 5′-overhangs;
b) incubating the resulting nucleic acid molecules with a DNA polymerase and nucleotide triphosphates to achieve complete filling of the 5′-overhangs, and therefore obtaining two double-stranded DNA copies of each target nucleic acid sequence; and
c) incubating the resulting nucleic acid molecules with a DNA ligase to achieve blunt-end ligation of the two double-stranded oligonucleotide copies of each target nucleic acid sequence of the starting repertoire in head-to-tail orientation.

7. The method of claim 6, wherein step c) is followed by:
d) amplifying the resulting ligated repertoire of target nucleic acid sequences by transformation of a host cell with said repertoire.

8. The method of claim 6, wherein step c) is followed by:
d) amplifying the resulting ligated repertoire of target nucleic acid sequences by polymerase chain reaction with oligonucleotide primers encompassing the target nucleic acid sequences, purification of the amplified DNA product, cloning into a double-stranded replicon, and transformation of a host cell with the ligated product.

9. The method of claim 6, wherein steps a), b) and c) are repeated at least one further cycle, such that the starting repertoire in step a) of a further cycle includes the resulting repertoire from step c) of a previous cycle, and the at least one further cycle forms a further starting repertoire for step a) of a still further cycle; such that the product of each concatenation cycle comprising steps a), b) and c), comprises a head-to-tail duplication of each of the target nucleic acid sequences in the starting repertoire, or a head-to-tail duplication of each of the target nucleic acid sequences in the repertoire obtained after step c) of a previous concatenation cycle.

10. The method of claim 6, wherein the single-stranded nicks define the 5′ ends of the target nucleic acid sequences on the top and bottom strands of target nucleic acid molecules.

11. The method of claim 6, wherein step a) is achieved by the action of a site-specific nicking endonuclease.

12. The method of claim 11, wherein the site-specific nicking endonuclease is N.BstNBI, and the target nucleic acid sequences are surrounded by two N.BstNBI recognition sequences which are oriented in opposite direction and located such that the distance between the 3′ side of each of the recognition sequence and said target sequences extends over 4 base pairs.

13. The method of claim 9, wherein the number of concatenation cycles is 2 to 4.

14. The method of claim 6, wherein said DNA polymerase exhibits strand-displacement activity.

15. The method of claim 14, wherein said DNA polymerase is Kienow fragment DNA polymerase I, Vent DNA polymerase, or Vent (exo) DNA polymerase.

16. A method for preparing concatenated polypeptides, comprising the steps of:
a. creating a concatenated repertoire of target nucleic acid sequences by a method according to claim 4;
b. translating the concatenated repertoire of target nucleic acid sequence to produce a repertoire of encoded concatenated polypeptides.

17. The method of claim 16 further comprising the step of selecting an encoded concatenated polypeptide having a desired activity.

18. The method according to claim 16, wherein each encoded concatenated polypeptide of the repertoire is expressed as a fusion protein.

19. The method according to claim 18, wherein each encoded concatenated polypeptide of the repertoire is expressed fused to a surface component of an organism so that each organism in a population thereof displays a concatenated polypeptide at its surface and comprises a concatenated nucleic acid encoding the displayed concatenated polypeptide.

20. The method according to claim 19 wherein the organism is a bacteriophage.

21. The method according to claim 16 wherein the concatenated target nucleic acid is used to express a concatenated polypeptide in a host cell.

22. The method according to claim 16 wherein the sequence of a concatenated target nucleic acid is used to derive a polypeptide by chemical synthesis.

23. The method according to claim 4, wherein the nucleic acids are further manipulated at the nucleic acid level.

24. The method according to claim 23, wherein the nucleic acids are manipulated by a technique selected from the group consisting of mutagenesis, fusion, insertion, truncation and derivatisation.

25. The method of claim 16 wherein the nucleic acids and/or polypeptides are further manipulated at the nucleic acid or protein level.

26. The method according to claim 25, wherein the nucleic acids are manipulated by a technique selected from the group consisting of mutagenesis, fusion, insertion, truncation and derivatisation.
